(19) United States
(12) Patent Application Publication
(10) Pub. No.: US 2022/0288133 A1
(43) Pub. Date: Sep. 15, 2022

(54) COMPOSITIONS FOR THE TREATMENT OF CONDITIONS BY DERMAL FILLERS

(71) Applicant: DERMATA THERAPEUTICS, LLC, San Diego, CA (US)

(72) Inventors: Gerald Thomas PROEHL, San Diego, CA (US); Christopher Joseph NARDO, San Diego, CA (US)

(21) Appl. No.: 17/628,810

(22) PCT Filed: Jul. 17, 2020
(86) PCT No.: PCT/US2020/042641

§ 371 Date: Jan. 20, 2022

Related U.S. Application Data
(60) Provisional application No. 62/877,680, filed on Jul. 23, 2019.

(51) Int. Cl. A61K 35/614; A61K 31/575; A61K 47/02; A61K 47/36; A61K 9/00; A61K 9/06; A61L 26/00; A61L 27/36; A61L 27/54; A61L 27/52

(52) U.S. Cl. CPC A61K 35/614; A61K 31/575; A61K 47/02; A61K 47/36; A61K 9/0014; A61K 9/06; A61L 26/0057; A61L 26/0066; A61L 26/008; A61L 27/3604; A61L 27/54; A61L 27/52

(57) ABSTRACT

The present disclosure relates to the treatment of diseases or conditions in a subject, including skin conditions or diseases, comprising applying to the skin of the subject a first composition comprising Spongilla, and a second composition comprising an effective amount of one or more dermal fillers, wherein said dermal fillers are selected from a chemical compound, a mixture of chemical compounds, a biological macromolecule, or an extract made from biological materials. Further provided are such methods wherein the one or more dermal fillers is selected from a group comprising of commercially available dermal fillers.

### CROSS REFERENCED APPLICATIONS

This application claims priority to U.S. Provisional Application No. 62/877,680 filed Jul. 23, 2019, the entirety of which is incorporated herein by reference.

### FIELD

The present disclosure relates to the cosmetic, reparative, and restorative dermalogical treatment of a subject, comprising applying to the skin of the subject first a composition comprising Spongilla, and a second composition comprising one or more dermal filler compositions including polymers and/or co-polymers of hyaluranonic acid, calcium hydroxylapatite, polyalkylimide, polymethylmethacrylate, collagens, polymethyl-methacrylate microspheres (PMMA), polylactic acid, polycaprolactone, carboxymethyl cellulose, poly-L-lactic acid, and cross-linking reagents. The disclosure also relates to products or kits for the cosmetic, reparative, and restorative dermalogical treatment of the subject's skin, comprising Spongilla, and a second composition comprising one or more dermal filler compositions. The present disclosure relates to the treatment of a condition in a subject, including a skin condition, comprising applying to the skin of the subject a first composition comprising Spongilla, and a second composition comprising one or more dermal filler compositions. The disclosure also relates to methods for delivering a dermal filler into the skin of a subject, including the dermis, comprising applying to the skin of the subject a first composition comprising Spongilla, and a second composition comprising an effective amount of dermal filler. The disclosure also relates to methods for delivering an effective amount of a dermal filler to an intradermal compartment of the subject's skin, such as the dermis, by applying to the skin of the subject a first composition comprising Spongilla, and a second composition comprising an effective amount of one or more dermal fillers. The disclosure also relates to methods for enhancing skin permeation of a topically applied dermal filler composition, comprising applying to the skin of the subject a first composition comprising Spongilla, followed by application of a second composition to the skin of the subject, wherein said second composition comprises an effective amount of said dermal filler composition, including polymers and/or co-polymers of hyaluranonic acid, calcium hydroxylapatite, polyalkylimide, polymethylmethacrylate, collagens, polymethyl-methacrylate microspheres (PMMA), polylactic acid, polycaprolactone, carboxymethyl cellulose, poly-L-lactic acid, and cross-linking reagents. Further, the second composition comprises an effective amount of a topical dermal filler composition, including glycerin, peptides derived from collagen, alguronic acid, low molecular weight hyaluronic acid polymers, sodium aceylated hyaluronate, glycolic acid, lactic acid, L-lactic acid, amino acids, NIA-114™, an acetyl peptide, Activen™ XEP-18, and/or Erasa™ XEP-30.

### BACKGROUND

Dermal fillers are mainly known for their cosmetic uses; however, dermal fillers can be used for medical purposes including treatment of human immunodeficiency virus-facial lipoatrophy. Despite the outstanding popularity of dermal fillers for dermatological applications, there has been little advancement in developing methods of delivery for these compounds to the area of interest for the patient. The higher molecular weight, hydrophilic polymers that comprise most commercial dermal fillers do not penetrate past the top layers of the skin. Therefore, most dermal fillers are delivered via needle injection in order to reach the desired subdermal depth and location. Traditional needle delivery has drawbacks, including causing anxiety in patients with a fear of needles, injection site pain, and injection site reactions to the high concentration of dermal filler (bolus injection). Therefore, there is a need for improved methods of treating or preventing skin conditions or diseases, and/or delivering a cosmetic application, in a subject using one or more dermal fillers in which the one or more dermal fillers is topically applied to the skin of the subject, avoids the drawbacks of needle-injection, that can lead to undesired side effects or adverse events, and permits an effective amount of the one or more dermal fillers to penetrate the skin of the subject so that an effective amount of the one or more dermal fillers is delivered to the desired site of action, such as the dermis of the subject.

### SUMMARY

The inventors of the subject matter disclosed herein have discovered that an important component of materials derived from Spongilla are the siliceous spicules that comprise the skeletal structure of Spongilla. The inventors have discovered the spicules can penetrate the stratum corneum and entire dermis of the skin of a subject during application and promote the penetration of topically-applied dermal fillers, such as a hydrogel composed of polymers, co-polymers, and cross-linked versions thereof into the skin of the subject, such as the dermis, to permit or improve the delivery of an effective amount of the dermal filler to the site of action necessary to effectively treat skin diseases or conditions in the subject. The inventors of the subject matter disclosed herein have also discovered that the spicules derived from Spongilla are useful in facilitating and permitting certain therapeutic compounds and compositions to penetrate into the skin of subjects to which the spicules are applied, which compounds and compositions would otherwise not be able to penetrate the skin of the subject in order to reach their targets and treat certain skin conditions. Among the compounds and compositions that may better penetrate the skin in the presence of materials derived from Spongilla are products comprising one or more chemical compounds, a mixture of chemical compounds, one or more biological macromolecules, or an extract made from biological materials. The inventors of the subject matter disclosed herein have discovered the materials comprising Spongilla used herein may comprise all organic and/or inorganic compounds and materials that are part of the naturally-occurring Spongilla, or may include only a portion of the organic and/or inorganic compounds and materials that are part of the naturally-occurring Spongilla.

In an aspect, provided herein are compositions including a first composition that includes Spongilla and a second composition that includes a dermal filler.

In an aspect, provided herein are methods for administering a dermal filler into the skin of a subject, the methods include applying to the skin of the subject a first composition that includes Spongilla, and a second composition that includes an effective amount of a dermal filler.

In an aspect, provided herein are methods for treating or preventing a skin condition or disease in a subject. The methods include administering to an intradermal compartment of the subject's skin, a therapeutically or prophylactically effective amount a first composition that includes Spongilla, and a second composition that includes a therapeutically effective amount a dermal filler. The methods include applying the first and second compositions to the skin of the subject.

In an aspect, provided herein are methods for enhancing skin permeation of a topically applied dermal filler composition into the skin of a subject. The methods include applying to the skin a first composition that includes Spongilla, followed by applying a second composition that includes an effective amount of a dermal filler.

In an aspect, provided herein are kits that include a first composition that includes Spongilla, and a second composition that includes one or more dermal fillers in an amount effective to treat a skin condition in a subject.

### BRIEF DESCRIPTION OF THE DRAWINGS

FIG. 1 shows the magnified surface of a typical Spongilla composition.

### DETAILED DESCRIPTION

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

As used herein, the term “about” means either within plus or minus 10% of the provided value, or rounded to the nearest significant figure, in all cases inclusive of the provided value. Where ranges are provided, they are inclusive of the boundary values.

As used herein, the terms “applied,” “applying,” “administration,” “administering,” and “used” means the delivery of a composition disclosed herein to a subject, in particular to the skin of the subject, by an administration route including, but not limited to, intraperitoneal, subcutaneous, intramuscular, topically, or any combinations thereof. In some embodiments disclosed herein, the compositions disclosed herein are administered to the subject, in particular to the skin of the subject, by topical administration.

As used herein, the term “aspect ratio” means with respect to the particles of Spongilla described herein the ratio between the average length of the particles to the average diameter of the particles.

As used herein, the terms “combination” and “in combination with” mean the application, use, or administration of one or more of the compositions disclosed herein, sequentially or simultaneously. It includes dosing simultaneously, or within minutes or hours of each other, or on the same day, or on alternating days, or using the compositions disclosed herein on a daily basis, or multiple days per week, or weekly basis, for example, while administering another composition on the same day or alternating days or weeks or on a periodic basis during a time simultaneous therewith or concurrent therewith, or at least a part of the time during which the composition disclosed herein is applied, used or administered. For example, one or more of the compositions disclosed herein, could be applied, used, or administered to a subject every day or several days a week while the additional composition is applied, used or dosed on alternating days or alternating weeks or other periods of time, such as every 1, 2, 3, 4, 5, 6, 7, 8, 9, 10, 11. 12, 13, 14 or more days.

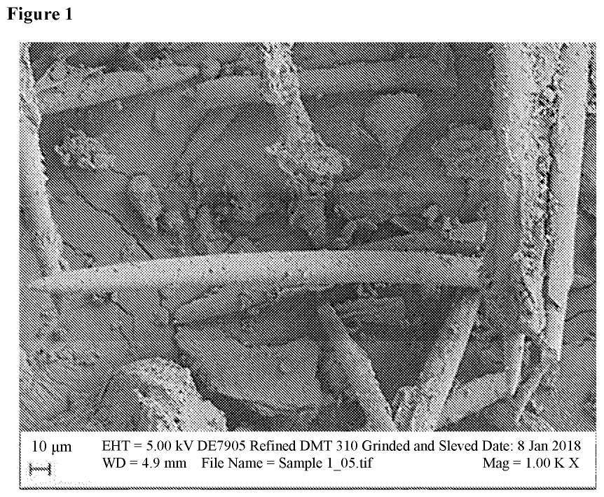

The term “Spongilla” as used herein means a genus of freshwater sponges in the family Spongillidae, including, but not limited to, Spongilla lacustris, S. fragilis Leidy, and Ephydatia fluviatilis. The term “Spongilla lacustris” as used herein means a species of sponge of the freshwater sponge family Spongillidae. FIG. 1 shows a scanning electron microscopy view of the structure of Spongillidae.

The terms “composition comprising Spongilla,” “powders comprising Spongilla, “materials comprising Spongilla, “Spongilla in the form of a powder,” and the like, as used herein, mean materials comprising Spongilla derived from raw Spongilla that is harvested and processed and may include all the various components of the Spongilla following harvest, including all organic and/or inorganic compounds and materials that are part of the naturally-occurring Spongilla, or may include only a portion of the organic and/or inorganic compounds and materials that are part of the naturally-occurring Spongilla. In one aspect is provided any of the methods or kits disclosed herein, wherein the Spongilla materials comprise all or substantially all the organic and inorganic materials derived from the naturally occurring Spongilla. In another aspect is provided any of the methods or kits disclosed herein, wherein the Spongilla materials comprise (a) only the spicules and any materials that are naturally associated with the spicules, or (b) substantially purified spicules and any materials that are naturally associated with the spicules, or (c) purified spicules and any materials that are naturally associated with the spicules that are a component part of naturally-occurring Spongilla. In another aspect is provided any of the methods or kits disclosed herein, wherein the Spongilla materials comprise only the spicules and any materials that are naturally associated with the spicules. In another aspect is provided any of the methods or kits disclosed herein, wherein the Spongilla materials comprise substantially purified spicules and any materials that are naturally associated with the spicules. In another aspect is provided any of the methods or kits disclosed herein, wherein the Spongilla materials comprise purified spicules and any materials that are naturally associated with the spicules that are a component part of naturally occurring Spongilla.

As used herein, the term “therapeutically effective amount” means that amount of the composition or combination of compositions being applied, used or administered to a subject that will treat, relieve, or prevent to some extent one or more of the symptoms of the disorder being treated.

### Compositions

In an aspect is provided a composition including a first composition and a second composition where the first composition includes Spongilla and the second composition includes one or more dermal fillers.

In embodiments, the first composition includes Spongilla. In embodiments, the Spongilla is Spongilla lacustris.

Spongilla, including Spongilla lacustris, and powders prepared from Spongilla that are utilized in the methods, uses, compositions for use as a medicament, kits and dermal fillers disclosed herein may be obtained, processed and characterized by methods known to those having ordinary skill in the art. For example, U.S. Pat. No. 7,604,821 describes the harvest, processing and characterization of several species of Spongilla, including Spongilla lacustris. The disclosure of U.S. Pat. No. 7,604,821 is incorporated herein by reference in its entirety. Sponge materials may be collected using methods commonly known to those skilled in the art of marine biology. For example, sponges can be collected manually using basic under water diving techniques, or in deeper waters larger colonies are harvested using the Agassiz trawl (AGT) or epibenthic sledge (EBS). Under certain environmental conditions, Spongilla colonies occur in a thin crust-like carpet several meters across and must be collected manually, with fork-like tools, and nets. The collected sponge mass is dried, cleaned of gross contamination, such as shells, stems, plants, rocks and other impurities, and is then washed to remove dirt, sand, silt and soluble impurities. The cleaned sponge mass is weighed and dried using methods known to those of ordinary skill in the art, such as air drying and the use of dryers that are used to dehydrate foods and pharmaceuticals. The sponge mass is dried until residual moisture content is less than a desired value as further disclosed herein. Residual moisture measurements can be performed using methods commonly known in the arts of food sciences, analytical chemistry or the pharmaceutical sciences. For example, 10 grams of dried material may be placed on a tared weighing boat and then weighed. The weighed material is then exposed to a heat source such as a drying oven or heat lamp operated at an appropriate temperature, the sample is then cooled in a desiccated chamber and re-weighed. Residual moisture is calculated as the percent difference between the sample weight before drying and the weight after cooling. Following drying, the sponge materials may be packaged in sealed containers, which optionally protect the materials from light, moisture and oxygen. The materials may then be further tested for the presence of pathogens, coliform organisms and organisms that represent a bioburden. The materials may be further heated or irradiated, as disclosed herein, to reduce any pathogens, coliform organisms or other organisms that represent a bioburden. The materials may then be further processed using methods known to those having ordinary skill in the art to provide a powder comprising particles having a desired size. For example, the sponge materials may be ground, and the resulting materials passed through one or more sieves of a defined size to provide a resulting material comprising particles having a uniform, or substantially uniform size. After final processing and sizing processes are completed, the dried sponge material may be packaged in airtight moisture-proof containers and stored at an appropriate temperature, such as at about room or ambient temperature.

In embodiments, the Spongilla is in the form of a powder. In embodiments, the powder includes particles that are substantially uniform in size.

In another aspect is provided any of the methods, compositions for use as a medicament, dermal fillers and kits disclosed herein, where the first composition includes Spongilla in the form of a powder. Materials include Spongilla may be prepared in powdered form having particles of substantially the same size, using techniques known to those having ordinary skill in the art, such as grinding and sieving. In another aspect is provided any of the methods, compositions for use as a medicament, dermal fillers and kits disclosed herein, where the Spongilla is in the form of a powder including particles that are substantially uniform in size. In another aspect is provided any of the methods, compositions for use as a medicament, dermal fillers and kits disclosed herein, where not less than about 50% of the particles include the Spongilla powder pass through a US 70-mesh screen. In another aspect is provided any of the methods, compositions for use as a medicament, dermal fillers and kits disclosed herein, where not less than about 60%, or about 70%, or about 75%, or about 80%, or about 85%, or about 90%, or about 95%, or about 96%, or about 97%, or about 98%, or about 99% of the particles comprising the Spongilla powder pass through a US 70-mesh screen. In another aspect is provided any of the methods, compositions for use as a medicament, dermal fillers and kits disclosed herein, where not less than about 95%, or about 96%, or about 97%, or about 98%, or about 99% of the particles comprising the Spongilla powder pass through a US 70-mesh screen. In another aspect is provided any of the methods, compositions for use as a medicament, dermal fillers and kits disclosed herein, where not less than about 95% of the particles including the Spongilla powder pass through a US 70-mesh screen. In another aspect is provided any of the methods, compositions for use as a medicament, dermal fillers and kits disclosed herein, wherein not less than about 96% of the particles including the Spongilla powder pass through a US 70-mesh screen. In another aspect is provided any of the methods, compositions for use as a medicament, dermal fillers and kits disclosed herein, where not less than about 97% of the particles including the Spongilla powder pass through a US 70-mesh screen. In another aspect is provided any of the methods, compositions for use as a medicament, dermal fillers and kits disclosed herein, where not less than about 98% of the particles including the Spongilla powder pass through a US 70-mesh screen. In another aspect is provided any of the methods, compositions for use as a medicament, dermal fillers and kits disclosed herein, where not less than about 99% of the particles including the Spongilla powder pass through a US 70-mesh screen. The particles of Spongilla may be manufactured or produced from Spongilla materials that are harvested by procedures known to those of ordinary skill in the art, such as determining the appropriate harvest period, removal of foreign materials, drying, milling and grinding using equipment known to those of ordinary skill in the art.

In another aspect is provided any of the methods, compositions for use as a medicament, dermal fillers and kits disclosed herein, where the particles comprising the Spongilla powder have an average length of from about 50 μm to about 500 μm. In another aspect is provided any of the methods, compositions for use as a medicament, dermal fillers and kits disclosed herein, where the particles comprising the Spongilla powder have an average length of from about 50 μm to about 400 μm, or from about 50 μm to about 350 μm, or from about 50 μm to about 300 μm, or from about 50 μm to about 250 μm, or from about 50 μm to about 200 μm, or from about 75 μm to about 500 μm, or from about 75 μm to about 450 μm, or from about 80 μm to about 450 μm, or from about 80 μm to about 400 μm, or from about 85 μm to about 450 μm, or from about 85 μm to about 400 μm, or from about 90 μm to about 450 μm, or from about 90 μm to about 400 μm, or from about 90 μm to about 350 μm, or from about 100 μm to about 450 μm, or from about 100 μm to about 400 μm, or from about 100 μm to about 350 μm, or from about 100 μm to about 300 μm, or from about 100 μm to about 250 μm, or from about 100 μm to about 200 μm, or from about 150 μm to about 500 μm, or from about 100 μm to about 450 μm, or from about 150 μm to about 400 μm, or from about 150 μm to about 350 μm, or from about 150 μm to about 350 μm, or from about 150 μm to about 300 μm, or from about 150 μm to about 250 μm, or from about 150 μm to about 200 μm, or from about 175 μm to about 450 μm, or from about 175 μm to about 400 μm, or from about 175 μm to about 350 μm, or from about 175 μm to about 300 μm, or from about 175 μm to about 250 μm, or from about 175 μm to about 200 μm. In another aspect is provided any of the methods, compositions for use as a medicament, dermal fillers and kits disclosed herein, where the particles comprising the Spongilla powder have an average length of about 50 μm, or about 75 μm, or about 80 μm, or about 85 μm, or about 90 μm, or about 100 μm, or about 125 μm, or about 150 μm, or about 175 μm, or about 200 μm, or about 225 μm, or about 250 μm, or about 300 μm, or about 350 μm, or about 400 μm, or about 450 μm, or about 500 μm. In another aspect is provided any of the methods, compositions for use as a medicament, dermal fillers and kits disclosed herein, where the particles comprising the Spongilla powder have an average length of about 200 μm. The particles comprising the Spongilla powder may be manufactured or produced from Spongilla materials that are harvested by procedures known to those of ordinary skill in the art, such as milling and grinding using equipment known to those of ordinary skill in the art. The average length of particles including the Spongilla powder may be measured using analytical methods known to those of ordinary skill in the art, such as, for example, scanning electron microscopy (SEM) and sieve analysis. Sieve analysis may also be used to determine the particle size distribution of the particles comprising the Spongilla powder.

In another aspect is provided any of the methods, compositions for use as a medicament, dermal fillers and kits disclosed herein, where the particles including the Spongilla powder have an average diameter of from about 5 μm to about 50 μm. In another aspect is provided any of the methods, compositions for use as a medicament, drug products and kits disclosed herein, where the particles including the Spongilla powder have an average diameter of from about 5 μm to about 45 μm, or from about 5 μm to about 40 μm, from about 5 μm to about 35 μm, from about 5 μm to about 30 μm, from about 5 μm to about 25 μm, from about 5 μm to about 20 μm, from about 10 μm to about 45 μm, from about 10 μm to about 40 μm, from about 10 μm to about 35 μm, from about 10 μm to about 30 μm, from about 10 μm to about 25 μm, from about 10 μm to about 20 μm, and from about 10 μm to about 15 μm. In another aspect is provided any of the methods, compositions for use as a medicament, drug products and kits disclosed herein, where the particles including the Spongilla powder have an average diameter of about 5 μm, or about 10 μm, or about 15 μm, or about 20 μm, or about 25 μm, or about 30 μm, or about 35 μm, or about 40 μm, or about 45 μm, or about 50 μm. The particles including the Spongilla powder may be manufactured or produced from Spongilla materials that are harvested by procedures known to those of ordinary skill in the art, such as milling and grinding using equipment known to those of ordinary skill in the art. The average diameter of particles comprising the Spongilla powder may be measured using analytical methods known to those of ordinary skill in the art, such as, for example, scanning electron microscopy (SEM) and sieve analysis. Sieve analysis may also be used to determine the particle size distribution of the particles comprising the Spongilla powder.

In another aspect is provided any of the methods, compositions for use as a medicament, dermal fillers and kits disclosed herein, where the particles including the Spongilla powder have an aspect ratio of from about 1 to about 100. In another aspect is provided any of the methods, compositions for use as a medicament, drug products and kits disclosed herein, where the particles including the Spongilla powder have an aspect ratio of from about 1 to about 75, or from about 1 to about 50, or from about 1 to about 25, or from about 1 to about 20, or from about 1 to about 15, or from about 5 to about 100, or from about 5 to about 75, or from about 5 to about 50, or from about 5 to about 40, or from about 5 to about 35, or from about 5 to about 30, or from about 5 to about 25, or from about 5 to about 20, or from about 5 to about 15, or from about 7 to about 50, or from about 7 to about 45, or from about 7 to about 40, or from about 7 to about 35, or from about 7 to about 30, or from about 7 to about 25, or from about 10 to about 50, or from about 10 to about 45, or from about 10 to about 40, or from about 10 to about 35, or from about 10 to about 30, or from about 10 to about 25, or from about 10 to about 15. In another aspect is provided any of the methods, compositions for use as a medicament, drug products and kits disclosed herein, where the particles including the Spongilla powder have an aspect ratio of about 5, or about 6, or about 7, or about 8, or about 9, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 35, or about 40, or about 45, or about 50, or about 75, or about 100. The particles including the Spongilla powder may be manufactured or produced from Spongilla materials that are harvested by procedures known to those of ordinary skill in the art, such as milling and grinding using equipment known to those of ordinary skill in the art. The aspect ratio of particles including the Spongilla powder may be measured using analytical methods known to those of ordinary skill in the art, such as, for example, scanning electron microscopy (SEM) and sieve analysis. Sieve analysis may also be used to determine the particle size distribution of the particles comprising the Spongilla powder.

Materials comprising Spongilla may be processed and dried, using techniques known to those having ordinary skill in the art, such as the use of drying ovens, to provide materials having a desired residual moisture content. In another aspect is provided any of the methods, compositions for use as a medicament, dermal fillers and kits disclosed herein, where the first composition including Spongilla has a residual moisture content of not more than about 20%. In another aspect is provided any of the methods, compositions for use as a medicament, drug products and kits disclosed herein, where the first composition has a residual moisture content of not more than about 15%, or not more than about 10%, or not more than about 9%, or not more than about 8%, or not more than about 7%, or not more than about 6%, or not more than about 5%, or not more than about 4%, or not more than about 3%, or not more than about 2%, or not more than 1%. In another aspect is provided any of the methods, compositions for use as a medicament, dermal fillers and kits disclosed herein, where the first composition has a residual moisture content of not more than about 5%. In another aspect is provided any of the methods, compositions for use as a medicament, dermal fillers and kits disclosed herein, where the first composition has a residual moisture content of not more than about 4%. In another aspect is provided any of the methods, compositions for use as a medicament, dermal fillers and kits disclosed herein, where the first composition has a residual moisture content of not more than about 3%. In another aspect is provided any of the methods, compositions for use as a medicament, dermal fillers and kits disclosed herein, where the first composition has a residual moisture content of not more than about 2%. In another aspect is provided any of the methods, compositions for use as a medicament, dermal fillers and kits disclosed herein, where the first composition has a residual moisture content of not more than about 1%. The moisture content of the Spongilla materials can be reduced by heating the raw Spongilla materials using methods known to those of ordinary skill in the art, such as by open-air drying, or by use of a conventional oven dryer or a vacuum dryer, using equipment known to those of ordinary skill in the art. For example, raw Spongilla materials may be placed into a tray and heated in a drying oven at a temperature range from about 30° C. to about 200° C., for example to about 70° C., for a period of time necessary to reduce the residual moisture content to the desired level. The level of residual moisture of the materials may be measured using methods known to those of ordinary skill in the art such as those described in the United States Pharmacopeia methods USP <731> (Loss on Drying) and USP <921> (Water Determination).

Materials comprising Spongilla may be treated in order to reduce the bioburden, such as aerobic and anaerobic microbes, yeast and mold, Coliform bacteria, Salmonella, Pseudomonas aeruginosa, and Staphylococcus aureus, of the materials prior to their packaging and use, such as by use of heat treatment or irradiation, such as the use of gamma irradiation. The materials comprising Spongilla maybe be treated after their packaging, such as by use of heat treatment or irradiation, such as the use of gamma radiation.

In another aspect is provided any of the methods and/or kits disclosed herein, wherein the first composition has a combined aerobic and anaerobic microbial content of not more than about 25×10⁴ colony-forming units per gram (CFU/g). In another aspect is provided any of the methods and/or kits disclosed herein, wherein the first composition has a combined aerobic and anaerobic microbial content of not more than about 10×10⁴ CFU/g, or not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods and/or kits disclosed herein, wherein the first composition has a combined aerobic and anaerobic microbial content of not more than about 1,000 CFU/g. In another aspect is provided any of the methods and/or kits disclosed herein, wherein the first composition has a combined aerobic and anaerobic microbial content of not more than about 750 CFU/g. In another aspect is provided any of the methods and/or kits disclosed herein, wherein the first composition has a combined aerobic and anaerobic microbial content of not more than about 500 CFU/g. In another aspect is provided any of the methods and/or kits disclosed herein, wherein the first composition has a combined aerobic and anaerobic microbial content of not more than about 250 CFU/g. In another aspect is provided any of the methods and/or kits disclosed herein, wherein the first composition has a combined aerobic and anaerobic microbial content of not more than about 200 CFU/g. In another aspect is provided any of the methods and/or kits disclosed herein, wherein the first composition has a combined aerobic and anaerobic microbial content of not more than about 150 CFU/g. In another aspect is provided any of the methods and/or kits disclosed herein, wherein the first composition has a combined aerobic and anaerobic microbial content of not more than about 100 CFU/g. In another aspect is provided any of the methods and/or kits disclosed herein, wherein the first composition has a combined aerobic and anaerobic microbial content of not more than about 75 CFU/g. In another aspect is provided any of the methods and/or kits disclosed herein, wherein the first composition has a combined aerobic and anaerobic microbial content of not more than about 50 CFU/g. In another aspect is provided any of the methods and/or kits disclosed herein, wherein the first composition has a combined aerobic and anaerobic microbial content of not more than about 25 CFU/g. In another aspect is provided any of the methods and/or kits disclosed herein, wherein the first composition has a combined aerobic and anaerobic microbial content of not more than about 20 CFU/g. In another aspect is provided any of the methods and/or kits disclosed herein, wherein the first composition has a combined aerobic and anaerobic microbial content of not more than about 10 CFU/g. In another aspect is provided any of the methods and/or kits disclosed herein, wherein the first composition has a combined aerobic and anaerobic microbial content of not more than about 1 CFU/g. The combined aerobic and anaerobic microbial content of the Spongilla materials may be reduced by physical or chemical methods and/or kits known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The combined aerobic and anaerobic microbial content of the Spongilla materials may be measured by methods known to those of ordinary skill in the art, such as those described in the United States Pharmacopeia method USP <61> (Microbial Enumeration Tests)

In another aspect is provided any of the methods and/or kits disclosed herein, wherein the first composition comprising Spongilla has a combined yeast and mold content of not more than about 25×10⁴ colony-forming units per gram (CFU/g). In another aspect is provided any of the methods and/or kits disclosed herein, wherein the first composition has a combined yeast and mold content of not more than about or not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods and/or kits disclosed herein, wherein the first composition has a combined yeast and mold content of not more than about 1,000 CFU/g. In another aspect is provided any of the methods and/or kits disclosed herein, wherein the first composition has a combined yeast and mold content of not more than about 750 CFU/g. In another aspect is provided any of the methods and/or kits disclosed herein, wherein the first composition has a combined yeast and mold content of not more than about 500 CFU/g. In another aspect is provided any of the methods and/or kits disclosed herein, wherein the first composition has a combined yeast and mold content of not more than about 250 CFU/g. In another aspect is provided any of the methods and/or kits disclosed herein, wherein the first composition has a combined yeast and mold content of not more than about 200 CFU/g. In another aspect is provided any of the methods and/or kits disclosed herein, wherein the first composition has a combined yeast and mold content of not more than about 150 CFU/g. In another aspect is provided any of the methods and/or kits disclosed herein, wherein the first composition has a combined yeast and mold content of not more than about 100 CFU/g. In another aspect is provided any of the methods and/or kits disclosed herein, wherein the first composition has a combined yeast and mold content of not more than about 75 CFU/g. In another aspect is provided any of the methods and/or kits disclosed herein, wherein the first composition has a combined yeast and mold content of not more than about 50 CFU/g. In another aspect is provided any of the methods and/or kits disclosed herein, wherein the first composition has a combined yeast and mold content of not more than about 25 CFU/g. In another aspect is provided any of the methods and/or kits disclosed herein, wherein the first composition has a combined yeast and mold content of not more than about 20 CFU/g. In another aspect is provided any of the methods and/or kits disclosed herein, wherein the first composition has a combined yeast and mold content of not more than about 10 CFU/g. In another aspect is provided any of the methods and/or kits disclosed herein, wherein the first composition has a combined yeast and mold content of not more than about 1 CFU/g. The combined yeast and mold content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The combined yeast and mold content of the Spongilla materials may be measured by methods known to those of ordinary skill in the art, such as those described in the United States Pharmacopeia method USP <61> (Microbial Enumeration Tests).

In another aspect is provided any of the methods and/or kits disclosed herein, wherein the amount of Salmonella in the first composition is not more than about 25×10⁴ colony-forming units per gram (CFU/g). In another aspect is provided any of the methods and/or kits disclosed herein, wherein the amount of Salmonella in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods and/or kits disclosed herein, wherein the first composition has a Salmonella content of not more than about 1,000 CFU/g. In another aspect is provided any of the methods and/or kits disclosed herein, wherein the first composition has a Salmonella content of not more than about 750 CFU/g. In another aspect is provided any of the methods and/or kits disclosed herein, wherein the first composition has a Salmonella content of not more than about 500 CFU/g. In another aspect is provided any of the methods and/or kits disclosed herein, wherein the first composition has a Salmonella content of not more than about 250 CFU/g. In another aspect is provided any of the methods and/or kits disclosed herein, wherein the first composition has a Salmonella content of not more than about 200 CFU/g. In another aspect is provided any of the methods and/or kits disclosed herein, wherein the first composition has a Salmonella content of not more than about 150 CFU/g. In another aspect is provided any of the methods and/or kits disclosed herein, wherein the first composition has a Salmonella content of not more than about 100 CFU/g. In another aspect is provided any of the methods and/or kits disclosed herein, wherein the first composition has a Salmonella content of not more than about 75 CFU/g. In another aspect is provided any of the methods and/or kits disclosed herein, wherein the first composition has a Salmonella content of not more than about 50 CFU/g. In another aspect is provided any of the methods and/or kits disclosed herein, wherein the first composition has a Salmonella content of not more than about 25 CFU/g. In another aspect is provided any of the methods and/or kits disclosed herein, wherein the first composition has a Salmonella content of not more than about 20 CFU/g. In another aspect is provided any of the methods and/or kits disclosed herein, wherein the first composition has a Salmonella content of not more than about 10 CFU/g. In another aspect is provided any of the methods and/or kits disclosed herein, wherein the first composition has a Salmonella content of not more than about 1 CFU/g. In another aspect is provided any of the methods and/or kits disclosed herein, wherein the first composition has no detectable Salmonella content. The Salmonella content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Salmonella content of the Spongilla materials may be measured by methods known to those of ordinary skill in the art, such as those described in the United States Pharmacopeia method USP <62> (Tests for Specified Microorganisms).

In another aspect is provided any of the methods and/or kits disclosed herein, wherein the amount of Coliform bacteria in the first composition is not more than about 25×10⁴ colony-forming units per gram (CFU/g). In another aspect is provided any of the methods and/or kits disclosed herein, wherein the amount of Coliform bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods and/or kits disclosed herein, wherein the first composition has a Coliform bacteria content of not more than about 1,000 CFU/g. In another aspect is provided any of the methods and/or kits disclosed herein, wherein the first composition has a Coliform bacteria content of not more than about 750 CFU/g. In another aspect is provided any of the methods and/or kits disclosed herein, wherein the first composition has a Coliform bacteria content of not more than about 500 CFU/g. In another aspect is provided any of the methods and/or kits disclosed herein, wherein the first composition has a Coliform bacteria content of not more than about 250 CFU/g. In another aspect is provided any of the methods and/or kits disclosed herein, wherein the first composition has a Coliform bacteria content of not more than about 200 CFU/g. In another aspect is provided any of the methods and/or kits disclosed herein, wherein the first composition has a Coliform bacteria content of not more than about 150 CFU/g. In another aspect is provided any of the methods and/or kits disclosed herein, wherein the first composition has a Coliform bacteria content of not more than about 100 CFU/g. In another aspect is provided any of the methods and/or kits disclosed herein, wherein the first composition has a Coliform bacteria content of not more than about 75 CFU/g. In another aspect is provided any of the methods and/or kits disclosed herein, wherein the first composition has a Coliform bacteria content of not more than about 50 CFU/g. In another aspect is provided any of the methods and/or kits disclosed herein, wherein the first composition has a Coliform bacteria content of not more than about 25 CFU/g. In another aspect is provided any of the methods and/or kits disclosed herein, wherein the first composition has a Coliform bacteria content of not more than about 20 CFU/g. In another aspect is provided any of the methods and/or kits disclosed herein, wherein the first composition has a Coliform bacteria content of not more than about 10 CFU/g. In another aspect is provided any of the methods and/or kits disclosed herein, wherein the first composition has a Coliform bacteria content of not more than about 1 CFU/g. In another aspect is provided any of the methods and/or kits disclosed herein, wherein the first composition has no detectable Coliform bacterial content. The Coliform bacteria content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Coliform bacteria content of the Spongilla materials may be measured by methods known to those of ordinary skill in the art, such as those described in the United States Pharmacopeia method USP <62> (Tests for Specified Microorganisms).

In another aspect is provided any of the methods and/or kits disclosed herein, wherein the amount of Pseudomonas aeruginosa bacteria in the first composition is not more than about 25×10⁴ colony-forming units per gram (CFU/g). In another aspect is provided any of the methods and/or kits disclosed herein, wherein the amount of Pseudomonas aeruginosa bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods and/or kits disclosed herein, wherein the first composition has a Pseudomonas aeruginosa bacteria content of not more than about 1,000 CFU/g. In another aspect is provided any of the methods and/or kits disclosed herein, wherein the first composition has a Pseudomonas aeruginosa bacteria content of not more than about 750 CFU/g. In another aspect is provided any of the methods and/or kits disclosed herein, wherein the first composition has a Pseudomonas aeruginosa bacteria content of not more than about 500 CFU/g. In another aspect is provided any of the methods and/or kits disclosed herein, wherein the first composition has a Pseudomonas aeruginosa bacteria content of not more than about 250 CFU/g. In another aspect is provided any of the methods and/or kits disclosed herein, wherein the first composition has a Pseudomonas aeruginosa bacteria content of not more than about 200 CFU/g. In another aspect is provided any of the methods and/or kits disclosed herein, wherein the first composition has a Pseudomonas aeruginosa bacteria content of not more than about 150 CFU/g. In another aspect is provided any of the methods and/or kits disclosed herein, wherein the first composition has a Pseudomonas aeruginosa bacteria content of not more than about 100 CFU/g. In another aspect is provided any of the methods and/or kits disclosed herein, wherein the first composition has a Pseudomonas aeruginosa bacteria content of not more than about 75 CFU/g. In another aspect is provided any of the methods and/or kits disclosed herein, wherein the first composition has a Pseudomonas aeruginosa bacteria content of not more than about 50 CFU/g. In another aspect is provided any of the methods and/or kits disclosed herein, wherein the first composition has a Pseudomonas aeruginosa bacteria content of not more than about 25 CFU/g. In another aspect is provided any of the methods and/or kits disclosed herein, wherein the first composition has a Pseudomonas aeruginosa bacteria content of not more than about 20 CFU/g. In another aspect is provided any of the methods and/or kits disclosed herein, wherein the first composition has a Pseudomonas aeruginosa bacteria content of not more than about 10 CFU/g. In another aspect is provided any of the methods and/or kits disclosed herein, wherein the first composition has a Pseudomonas aeruginosa bacteria content of not more than about 1 CFU/g. In another aspect is provided any of the methods and/or kits disclosed herein, wherein the first composition has no detectable Pseudomonas aeruginosa bacteria content. The Pseudomonas aeruginosa bacteria content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Pseudomonas aeruginosa bacteria content of the Spongilla materials may be measured by methods known to those of ordinary skill in the art, such as those described in the United States Pharmacopeia method USP <62> (Tests for Specified Microorganisms).

In another aspect is provided any of the methods and/or kits disclosed herein, wherein the amount of Staphylococcus aureus bacteria in the first composition is not more than about 25×10⁴ colony-forming units per gram (CFU/g). In another aspect is provided any of the methods and/or kits disclosed herein, wherein the amount of Staphylococcus aureus bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another aspect is provided any of the methods and/or kits disclosed herein, wherein the first composition has a Staphylococcus aureus bacteria content of not more than about 1,000 CFU/g. In another aspect is provided any of the methods and/or kits disclosed herein, wherein the first composition has a Staphylococcus aureus bacteria content of not more than about 750 CFU/g. In another aspect is provided any of the methods and/or kits disclosed herein, wherein the first composition has a Staphylococcus aureus bacteria content of not more than about 500 CFU/g. In another aspect is provided any of the methods and/or kits disclosed herein, wherein the first composition has a Staphylococcus aureus bacteria content of not more than about 250 CFU/g. In another aspect is provided any of the methods and/or kits disclosed herein, wherein the first composition has a Staphylococcus aureus bacteria content of not more than about 200 CFU/g. In another aspect is provided any of the methods and/or kits disclosed herein, wherein the first composition has a Staphylococcus aureus bacteria content of not more than about 150 CFU/g. In another aspect is provided any of the methods and/or kits disclosed herein, wherein the first composition has a Staphylococcus aureus bacteria content of not more than about 100 CFU/g. In another aspect is provided any of the methods and/or kits disclosed herein, wherein the first composition has a Staphylococcus aureus bacteria content of not more than about 75 CFU/g. In another aspect is provided any of the methods and/or kits disclosed herein, wherein the first composition has a Staphylococcus aureus bacteria content of not more than about 50 CFU/g. In another aspect is provided any of the methods and/or kits disclosed herein, wherein the first composition has a Staphylococcus aureus bacteria content of not more than about 25 CFU/g. In another aspect is provided any of the methods and/or kits disclosed herein, wherein the first composition has a Staphylococcus aureus bacteria content of not more than about 20 CFU/g. In another aspect is provided any of the methods and/or kits disclosed herein, wherein the first composition has a Staphylococcus aureus bacteria content of not more than about 10 CFU/g. In another aspect is provided any of the methods and/or kits disclosed herein, wherein the first composition has a Staphylococcus aureus bacteria content of not more than about 1 CFU/g. In another aspect is provided any of the methods and/or kits disclosed herein, wherein the first composition has no detectable Staphylococcus aureus bacteria content. The Staphylococcus aureus bacteria content of the Spongilla materials may be reduced by physical or chemical methods known to those of ordinary skill in the art, such as physical treatment of the materials with heat in the form of steam or dry heat, or chemical treatment in the form of exposure to ethylene oxide gas or treatment by ionizing radiation for a sufficient amount of time to reduce the microbial content to the desired levels. The Staphylococcus aureus bacteria content of the Spongilla materials may be measured by methods known to those of ordinary skill in the art, such as those described in the United States Pharmacopeia method USP <62> (Tests for Specified Microorganisms).

In another aspect is provided any of the methods, compositions for use as a medicament, dermal fillers and kits disclosed herein, where the first composition is packaged prior to use. In another aspect is provided any of the methods, compositions for use as a medicament, dermal fillers and kits disclosed herein, where the first composition is prepared by heating to at least about 70° C. prior to being packaged in order to reduce the bioburden. In another aspect is provided any of the methods, compositions for use as a medicament, dermal fillers and kits disclosed herein, where the first composition is prepared by heating to at least about 50° C., or at least about 60° C., or at least about 75° C., or at least about 80° C., or at least about 85° C., or at least about 90° C., or at least about 100° C., or at least about 110° C., or at least about 115° C., or at least about 120° C., or at least about 125° C., or at least about 130° C., or at least about 135° C., or at least about 140° C., or at least about 150° C., or at least about 160° C., or at least about 170° C., or at least about 180° C., or at least about 190° C., or at least about 200° C. prior to being packaged.

In another aspect is provided any of the methods, compositions for use as a medicament, dermal fillers and kits disclosed herein, where the first composition comprising Spongilla is heated to at least about 70° C. for at least about 5 minutes prior being packaged. In another aspect is provided any of the methods, compositions for use as a medicament, dermal fillers and kits disclosed herein, where the first composition is heated to at least about 70° C. for at least about 10 minutes, or at least about 15 minutes, or at least about 20 minutes, or at least about 25 minutes, or at least about 30 minutes, or at least about 35 minutes, or at least about 40 minutes, or at least about 45 minutes, or at least about 50 minutes, or at least about 55 minutes, or at least about 60 minutes, or at least about 75 minutes, or at least about 90 minutes, or at least about 120 minutes, or at least about 180 minutes, or at least about 4 hours, or at least about 5 hours, or at least about 6 hours, or at least about 7 hours, or at least about 8 hours, or at least about 9 hours, or at least about 10 hours, or at least about 11 hours, or at least about 12 hours, or at least about 24 hours prior being packaged.

In another aspect is provided any of the methods, compositions for use as a medicament, dermal fillers and kits disclosed herein, where the first composition including Spongilla is prepared by treating with ionizing radiation, such as gamma radiation, prior to being packaged or after packaging. For example, gamma irradiation may be performed on the raw Spongilla material prior to grinding to reduce the particle size, following grinding to reduce the particle size, the materials packaged in bulk and or the materials following packaging in unit dose containers. The materials may be treated with ionizing radiation, such as gamma radiation, using methods and equipment known to those of ordinary skill in the art, such as gamma irradiators or electron beam irradiators. In another aspect is provided any of the methods, compositions for use as a medicament, dermal fillers and kits disclosed herein, wherein the first composition is prepared by treating with ionizing radiation, such as gamma radiation, to deliver an absorbed radiation dose between about 1 kGy and about 50 kGy prior to being packaged. In another aspect is provided any of the methods, compositions for use as a medicament, dermal fillers and kits disclosed herein, where the first composition is prepared by treating with ionizing radiation, such as gamma radiation, to deliver an absorbed radiation dose between about 1 kGy and about 45 kGy, or between about 1 kGy and about 40 kGy, between about 1 kGy and about 35 kGy, between about 1 kGy and about 30 kGy, or between about 1 kGy and about 25 kGy or between about 5 kGy and about 50 kGy, or between about 5 kGy and about 45 kGy, or between about 5 kGy and about 40 kGy, or between about 5 kGy and about 35 kGy, or between about 5 kGy and about 30 kGy, or between about 5 kGy and about 25 kGy, or between about 10 kGy and about 50 kGy, or between about 10 kGy and about 45 kGy, or between about 10 kGy and about 40 kGy, or between about 10 kGy and about 35 kGy, or between about 10 kGy and about 30 kGy, or between about 10 kGy and about 25 kGy, or between about 15 kGy and about 50 kGy, or between about 15 kGy and about 45 kGy, or between about 15 kGy and about 40 kGy, or between about 15 kGy and about 35 kGy, or between about 15 kGy and about 30 kGy, or between about 15 kGy and about 25 kGy. In another aspect is provided any of the methods, compositions for use as a medicament, dermal fillers and kits disclosed herein, wherein the first composition is prepared by treating with ionizing radiation, such as gamma radiation, to deliver an absorbed radiation dose of about 1 kGy, or about 5 kGy, or about 10 kGy, 11 kGy, or about 12 kGy, or about 13 kGy, or about 14 kGy, or about 15 kGy, or about 16 kGy, or about 17 kGy, or about 18 kGy, or about 19 kGy, or about 20 kGy, or about 21 kGy, or about 22 kGy, or about 23 kGy, or about 24 kGy, or about 25 kGy, or about 26 kGy, or about 27 kGy, or about 28 kGy, or about 29 kGy, or about 30 kGy, or about 31 kGy, or about 32 kGy, or about 33 kGy, or about 34 kGy, or about 35 kGy, or about 36 kGy, or about 37 kGy, or about 38 kGy, or about 39 kGy, or about 40 kGy, or about 41 kGy, or about 42 kGy, or about 43 kGy, or about 44 kGy, or about 45 kGy, or about 46 kGy, or about 47 kGy, or about 48 kGy, or about 49 kGy, or about 50 kGy.

In another aspect is provided any of the methods, compositions for use as a medicament, dermal fillers and kits disclosed herein, where the Spongilla includes Spongilla lacustris.

In another aspect is provided any of the methods, compositions for use as a medicament, dermal fillers and kits disclosed herein, where the amount of the first composition including Spongilla applied to the skin of the subject is from about 0.5 grams to about 50 grams. In one aspect is provided any of the methods disclosed herein, where the amount of the first composition is measured as a dry weight. In another aspect is provided any of the methods, compositions for use as a medicament, dermal fillers and kits disclosed herein, where the amount of the first composition including Spongilla applied to the skin of the subject is from about 0.5 grams to about 40 grams, or from about 0.5 grams to about 35 grams, or from about 0.5 grams to about 30 grams, or from about 0.5 grams to about 25 grams, or from about 0.5 grams to about 20 grams, or from about 0.5 grams to about 15 grams, or from about 0.5 grams to about 10 grams, or from about 0.75 grams to about 20 grams, or from about 0.75 grams to about 15 grams, or from about 0.75 grams to about 10 grams, or from about 1 gram to about 20 grams, or from about 1 gram to about 15 grams, or from about 1 gram to about 10 grams, or from about 1 gram to about 9 grams, or from about 1 gram to about 8 grams, or from about 1 gram to about 7 grams, or from about 1 gram to about 6 grams, or from about 1 gram to about 5 grams, or from about 1 gram to about 4 grams, or from about 1 gram to about 3 grams, or from about 1 gram to 2 grams. In another aspect is provided any of the methods, compositions for use as a medicament, dermal fillers and kits disclosed herein, where the amount of Spongilla applied to the skin, such as those disclosed above, are each measured as a dry weight.

In another aspect is provided any of the methods, compositions for use as a medicament, dermal fillers products and kits disclosed herein, where the amount of the first composition including Spongilla applied to the skin of the subject is about 0.5 grams, or about 0.75 grams, or about 1 gram, or about 1.25 grams, or about 1.5 grams, or about 1.75 grams, or about 2 grams, or about 2.25 grams, or about 2.5 grams, or about 2.75 grams, or about 3 grams, or about 3.25 grams, or about 3.5 grams, or about 3.75 grams, or about 4 grams, or about 4.25 grams, or about 4.5 grams, or about 4.75 grams, or about 5 grams, or about 5.25 grams, or about 5.5 grams, or about 5.75 grams, or about 6 grams, or about 6.25 grams, or about 6.5 grams, or about 7 grams, or about 7.25 grams, or about 7.5 grams, or about 7.75 grams, or about 8 grams, or about 8.25 grams, or about 8.5 grams, or about 8.75 grams, or about 9 grams, or about 9.25 grams, or about 9.5 grams, or about 9.75 grams, or about 10 grams, or about 11 grams, or about 12 grams, or about 13 grams, or about 14 grams, or about 15 grams, or about 16 grams, or about 17 grams, or about 18 grams, or about 19 grams, or about 20 grams, or about 25 grams, or about 35 grams, or about 40 grams, or about 45 grams, or about 50 grams, or about 75 grams, or about 100 grams, or about 250 grams, or about 500 grams, or about 750 grams, or about 1000 grams, in each case measured as a dry weight.

In another aspect is provided any of the methods, compositions for use as a medicament, drug products and kits disclosed herein, wherein the first composition is applied to the skin of the subject in the form of a paste. In another aspect is provided any of the methods, compositions for use as a medicament, drug products and kits disclosed herein, wherein the paste further comprises water or saline. In another aspect is provided any of the methods, compositions for use as a medicament, drug products and kits disclosed herein, wherein the paste is prepared by mixing a composition comprising Spongilla and an aqueous solution comprising hydrogen peroxide. In another aspect is provided any of the methods, compositions for use as a medicament, drug products and kits disclosed herein, wherein the hydrogen peroxide is at a concentration of from about 0.1% w/w to about 50% w/w, or from about 0.1% w/w to about 45% w/w, or from about 0.1% w/w to about 40% w/w, or from about 0.1% w/w to about 35% w/w, or from about 0.1% w/w to about 30% w/w, or from about 0.1% w/w to about 25% w/w, or from about 0.1% w/w to about 20% w/w, or from about 0.1% w/w to about 15% w/w, or from about 0.1% w/w to about 10% w/w, or from about 0.1% w/w to about 9% w/w, or from about 0.1% w/w to about 8% w/w, or from about 0.1% w/w to about 8% w/w, or from about 0.1% w/w to about 7% w/w, or from about 0.1% w/w to about 6% w/w, or from about 0.1% w/w to about 5% w/w, or from about 0.1% w/w to about 4% w/w, or from about 0.1% w/w to about 3% w/w, or from about 0.1% w/w to about 2% w/w, or from about 0.1% w/w to about 1% w/w, or from about 0.5% w/w to about 45% w/w, or from about 1% w/w to about 45% w/w, or from about 1% w/w to about 40% w/w, or from about 1% w/w to about 35% w/w, or from about 1% w/w to about 30% w/w, or from about 1% w/w to about 25% w/w, or from about 1% w/w to about 20% w/w, or from about 1% w/w to about 15% w/w, or from about 1% w/w to about 10% w/w, or from about 1% w/w to about 9% w/w, or from about 1% w/w to about 8% w/w, or from about 1% w/w to about 8% w/w, or from about 1% w/w to about 7% w/w, or from about 1% w/w to about 6% w/w, or from about 1% w/w to about 5% w/w, or from about 1% w/w to about 4% w/w, or from about 1% w/w to about 3% w/w, or from about 1% w/w to about 2% w/w, or from about 2% w/w to about 45% w/w, or from about 2% w/w to about 40% w/w, or from about 2% w/w to about 35% w/w, or from about 2% w/w to about 30% w/w, or from about 2% w/w to about 25% w/w, or from about 2% w/w to about 20% w/w, or from about 2% w/w to about 15% w/w, or from about 2% w/w to about 10% w/w, or from about 2% w/w to about 9% w/w, or from about 2% w/w to about 8% w/w, or from about 1% w/w to about 7% w/w, or from about 2% w/w to about 6% w/w, or from about 2% w/w to about 5% w/w, or from about 2% w/w to about 4% w/w, or from about 2% w/w to about 3% w/w. In another aspect is provided any of the methods, compositions for use as a medicament, drug products and kits disclosed herein, wherein the hydrogen peroxide is at a concentration of about 0.1% w/w, or about 0.5% w/w, or about 1% w/w, or about 2% w/w, or about 3% w/w, or about 4% w/w, or about 5% w/w, or about 6% w/w, or about 7% w/w, or about 8% w/w, or about 9% w/w, or about 10% w/w, or about 15% w/w, or about 20% w/w, or about 25% w/w, or about 30% w/w, or about 35% w/w, or about 40% w/w, or about 45% w/w, or about 50% w/w. In another aspect is provided any of the methods, compositions for use as a medicament, drug products and kits disclosed herein, wherein the hydrogen peroxide is at a concentration of about 3% w/w. Aqueous hydrogen peroxide solutions that may be useful in treating skin conditions in a subject as disclosed herein are commercially available or may be prepared by methods known to those of ordinary skill in the art.

In another aspect is provided any of the methods, compositions for use as a medicament, drug products and kits disclosed herein, wherein the first composition and/or the second composition may be used in combination with a gel or cream, which gel or cream may or may not further comprise hydrogen peroxide. In another aspect is provided any of the methods, compositions for use as a medicament, drug products and kits disclosed herein, wherein the first composition and/or the second composition may be used in combination with a gel or cream, which gel or cream does not further comprise hydrogen peroxide. In another aspect is provided any of the methods, compositions for use as a medicament, drug products and kits disclosed herein, wherein the first composition and/or the second composition may be used in combination with a gel or cream, which gel or cream further comprises hydrogen peroxide. Such gels or creams are generally commercially available any may contain from about 0.5% w/w to about 50% w/w hydrogen peroxide. For example, a gel containing about 1% w/w, or about 2% w/w, or about 3% w/w, or about 4% w/w, or about 6% w/w, or about 7% w/w, or about 8% w/w, or about 9% w/w, or about 10% w/w, or about 15% w/w, or about 20% w/w, or about 25% w/w, or about 30% w/w, or about 40% w/w, or about 45% w/w, or about 50% w/w hydrogen peroxide may be used in any of the methods disclosed herein in combination with the first composition and the second composition.

In embodiments, the composition that includes one or more dermal fillers. In embodiments, the derm filler is selected from a chemical compound, a mixture of chemical compounds, a biological macromolecule, or an extract made from biological materials. In embodiments, the one or more dermal fillers is a chemical compound. In embodiments, the one or more dermal fillers is a biological macromolecule. In embodiments, the one or more dermal fillers is an extract made from biological materials.

In embodiments, a dermal filler includes polymers, co-polymers, cross-linked polymers, and/or cross-linked copolymers of hyaluranonic acid, calcium hydroxylapatite, polyalkylimide, polymethylmethacrylate, collagens, polymethyl-methacrylate microspheres (PMMA), polylactic acid, polycaprolactone, carboxymethyl cellulose, poly-L-lactic acid. In embodiments, a dermal filler includes polymers, co-polymers, cross-linked polymers, and/or cross-linked copolymers of hyaluranonic acid. In embodiments, a dermal filler includes polymers, co-polymers, cross-linked polymers, and/or cross-linked copolymers of calcium hydroxlyapatite. In embodiments, a dermal filler includes polymers, co-polymers, cross-linked polymers, and/or cross-linked copolymers of polyalkylimide. In embodiments, a dermal filler includes polymers, co-polymers, cross-linked polymers, and/or cross-linked copolymers of polymethylmethacrylate. In embodiments, a dermal filler includes polymers, co-polymers, cross-linked polymers, and/or cross-linked copolymers of collagen. In embodiments, a dermal filler includes polymers, co-polymers, cross-linked polymers, and/or cross-linked copolymers of polymethyl-methacrylate microspheres (PMMA). In embodiments, a dermal filler includes polymers, co-polymers, cross-linked polymers, and/or cross-linked copolymers of polylactic acid. In embodiments, a dermal filler includes polymers, co-polymers, cross-linked polymers, and/or cross-linked copolymers of polycaprolactone. In embodiments, a dermal filler includes polymers, co-polymers, cross-linked polymers, and/or cross-linked copolymers of carboxymethyl cellulose. In embodiments, a dermal filler includes polymers, co-polymers, cross-linked polymers, and/or cross-linked copolymers of poly-L-lactic acid.

In embodiments, a dermal filler includes a topical dermal filler, including glycerin, peptides derived from collagen, alguronic acid, low molecular weight hyaluronic acid polymers, sodium aceylated hyaluronate, glycolic acid, lactic acid, L-lactic acid, amino acids, NIA-114™, an acetyl peptide, Activen™ XEP-18, and/or Erasa™ XEP-30. In embodiments, a dermal filler includes glycerin. In embodiments, a dermal filler includes peptides derived from collagen. In embodiments, a dermal filler includes alguronic acid. In embodiments, a dermal filler includes low molecular weight hyaluronic acid polymers. In embodiments, a dermal filler includes sodium aceylated hyaluronate. In embodiments, a dermal filler includes glycolic acid. In embodiments, a dermal filler includes lactic acid. In embodiments, a dermal filler includes L-lactic acid. In embodiments, a dermal filler includes amino acids. In embodiments, a dermal filler includes NIA-114™. In embodiments, a dermal filler includes acetyl peptide. In embodiments, a dermal filler includes Activen™ XEP-18. In embodiments, a dermal filler includes Erasa™ XEP-30.

In embodiments, the first composition includes Spongilla and the second composition includes a dermal filler in an amount effective to treat a skin disease or condition in a subject, selected from one or more commercially available dermal fillers. In embodiments, the first composition includes Spongilla and the second composition includes Restylane®. In embodiments, the first composition includes Spongilla and the second composition includes Restylane® Lyft (Perlane®-L). In embodiments, the first composition includes Spongilla and the second composition includes Restylane® Refyne. In embodiments, the first composition includes Spongilla and the second composition includes Restylane® Defyne. In embodiments, the first composition includes Spongilla and the second composition includes Restylane® Silk. In embodiments, the first composition includes Spongilla and the second composition includes Belotero® Hydro. In embodiments, the first composition includes Spongilla and the second composition includes Belotero® Soft. In embodiments, the first composition includes Spongilla and the second composition includes Belotero® Balance. In embodiments, the first composition includes Spongilla and the second composition includes Captique™. In embodiments, the first composition includes Spongilla and the second composition includes Esthelis™. In embodiments, the first composition includes Spongilla and the second composition includes Fortelis™. In embodiments, the first composition includes Spongilla and the second composition includes Modelis™. In embodiments, the first composition includes Spongilla and the second composition includes Elevess® (Hydrelle®). In embodiments, the first composition includes Spongilla and the second composition includes Hylaform® (hylan B gel). In embodiments, the first composition includes Spongilla and the second composition includes Puragen™. In embodiments, the first composition includes Spongilla and the second composition includes Prevelle® Silk. In embodiments, the first composition includes Spongilla and the second composition includes Radiesse®. In embodiments, the first composition includes Spongilla and the second composition includes Radiesse® (+). In embodiments, the first composition includes Spongilla and the second composition includes Aquamid®. In embodiments, the first composition includes Spongilla and the second composition includes Sculptura®. In embodiments, the first composition includes Spongilla and the second composition includes Bellafill®. In embodiments, the first composition includes Spongilla and the second composition includes Juvaderm®.

In embodiments, the second composition includes deoxycholic acid. Deoxycholic acid is also known as cholanoic acid and 3α,12α-dihydroxy-5β-cholan-24-oic acid.

In embodiments, provided herein are a first composition including Spongilla, and a second composition including one or more dermal fillers in an amount effective to treat a disease or condition in a subject, including a skin condition or disease. In embodiments, provided herein is a kit including a first composition comprising Spongilla, and a second composition including one or more dermal fillers in an amount effective to treat a condition in a subject, including a skin condition. In embodiments, provided herein is a product including a first composition and a second composition, where the first composition includes Spongilla, and the second composition includes one or more dermal fillers in an amount effective to treat a skin disease or condition in a subject, and where the second composition is in the form of a solution, an aqueous solution, a powder, or a hydrogel.

In another embodiment is provided any of the dermal filler compositions or kits disclosed herein, where the disease or condition, including a skin disease or condition, is selected from lipoatrophy due to human immunodeficiency virus infection or acne scarring, facial rhytids, folds, depressions, augmentation of existing facial, body, or extremity structures, improvement of nasal function, and/or cheek augmentation. In another aspect is provided any of the dermal filler compositions or kits disclosed herein, where the disease or condition is selected from correction of fine, moderate, and/or severe wrinkles or folds of skin in the face, lips, nose, under-eye region, eye brow, head, chin, neck, ear, ear lobes, décolletage, elbow, hand, foot, and knee regions. In another aspect is provided any of the dermal filler compositions or kits disclosed herein, where the disease or condition is selected from to correction and/or modification of the shape of specific regions of face, lips, nose, under-eye region, eye brow, head, chin, neck, ear, ear lobes, décolletage, elbow, hand, foot, and knee regions. In another aspect is provided any of the dermal filler compositions or kits disclosed herein, where the disease or condition, including a skin disease or condition, is improvement of the appearance of moderate to severe convexity or fullness associated with submental adipose tissue, or fat.

In another embodiment is provided any of the dermal filler compositions or kits disclosed herein, where the disease or condition, including a skin disease or condition, is selected from lip augmentation, perioral rhytids, moderate to severe facial folds and wrinkles such as nasolabial folds, moderate to severe facial wrinkles such as smile lines or marionette lines, age-related midface contour deficiencies, dorsal hand to correct volume deficit, glabellar lines, correction of facila depressions, either due to injury or age-related, perioral wrinkles, lip commissures, crow's feet, forehead wrinkles, soft tissue cotour deficiencies, acne scars, moderate to severe, distensible facial acne scars on the cheek, age-related volume loss, facial lipoatrophy or fat wasting in HIV+ patients, and improvement of the appearance of moderate to severe convexity or fullness associated with submental adipose tissue, or fat. In another embodiment is provided any of the drug products or kits disclosed herein, wherein the disease or condition is lip augmentation. In another embodiment is provided any of the drug products or kits disclosed herein, wherein the disease or condition is perioral rhytids. In another embodiment is provided any of the drug products or kits disclosed herein, wherein the disease or condition is moderate to severe facial folds and wrinkles such as nasolabial folds. In another embodiment is provided any of the drug products or kits disclosed herein, wherein the disease or condition is moderate to severe facial wrinkles such as smile lines or marionette lines. In another embodiment is provided any of the drug products or kits disclosed herein, wherein the disease or condition is age-related midface contour deficiencies. In another embodiment is provided any of the drug products or kits disclosed herein, wherein the disease or condition is dorsal hand to correct volume deficit. In another embodiment is provided any of the drug products or kits disclosed herein, wherein the disease or condition is glabellar lines. In another embodiment is provided any of the drug products or kits disclosed herein, wherein the disease or condition is correction of facila depressions, either due to injury or age-related. In another embodiment is provided any of the drug products or kits disclosed herein, wherein the disease or condition is perioral wrinkles. In another embodiment is provided any of the drug products or kits disclosed herein, wherein the disease or condition is lip commissures. In another embodiment is provided any of the drug products or kits disclosed herein, wherein the disease or condition is crow's feet. In another embodiment is provided any of the drug products or kits disclosed herein, wherein the disease or condition is forehead wrinkles. In another embodiment is provided any of the drug products or kits disclosed herein, wherein the disease or condition is soft tissue cotour deficiencies. In another embodiment is provided any of the drug products or kits disclosed herein, wherein the disease or condition is acne scars. In another embodiment is provided any of the drug products or kits disclosed herein, wherein the disease or condition is moderate to severe, distensible facial acne scars on the cheek. In another embodiment is provided any of the drug products or kits disclosed herein, wherein the disease or condition is age-related volume loss. In another embodiment is provided any of the drug products or kits disclosed herein, wherein the disease or condition is facial lipoatrophy or fat wasting in HIV+ patients. In another embodiment is provided any of the drug products or kits disclosed herein, wherein the disease or condition is improvement of the appearance of moderate to severe convexity or fullness associated with submental adipose tissue, or fat.

In another embodiment is provided any of the dermal filler compositions or kits disclosed herein, wherein the one or more dermal fillers is selected from a chemical compound, a mixture of chemical compounds, a biological macromolecule, or an extract made from biological materials. In another aspect is provided any of the dermal filler compositions or kits disclosed herein, where said one or more dermal fillers is a biological macromolecule. In another aspect is provided any of the dermal filler compositions or kits disclosed herein, where the one or more dermal fillers is a chemical compound. In another aspect is provided any of the dermal filler compositions or kits disclosed herein, where the one or more deremal fillers is a mixture of chemical compounds. In another aspect is provided any of the dermal filler compostions or kits disclosed herein, where the one or more dermal fillers is a biological macromolecule. In another aspect is provided any of the dermal filler compositions or kits disclosed herein, where the one or more dermal fillers is an extract made from biological materials.

### Methods

In an aspect, provided herein are methods for treating or preventing one or more skin conditions or diseases in a subject including delivering a therapeutically or prophylactically effective amount of a dermal filler to an intradermal compartment of the subject's skin by applying to the skin of the subject a first composition including Spongilla, and a second composition comprising a therapeutically effective amount of one or more dermal fillers.

In embodiments, the disease or condition is selected from lipoatrophy, facial rhytids, folds, depressions, augmentation of existing facial, body, or extremity structures, or improvement of nasal function. In embodiments, the disease or condition is lipoatrophy. In embodiments, the lipoatrophy is due to human immunodeficiency virus infection or acne scarring. In embodiments, the disease or condition is facial rhytids. In embodiments, the disease or condition is facial skin folds. In embodiments, the disease or condition is skin folds. In embodiments, the disease or condition is treated by augmentation of an existing facial structure(s). In embodiments, the disease or condition is treated by augmentation of an existing body structure(s). In embodiments, the disease or condition is treated by augmentation of an existing extremity structure(s). In embodiments, the disease or condition is treated by improving nasal function.

In another embodiment the disease or condition is selected from lip augmentation, perioral rhytids, moderate to severe facial folds and wrinkles such as nasolabial folds, moderate to severe facial wrinkles such as smile lines or marionette lines, age-related midface contour deficiencies, dorsal hand to correct volume deficit, glabellar lines, correction of facila depressions, either due to injury or age-related, perioral wrinkles, lip commissures, crow's feet, forehead wrinkles, soft tissue contour deficiencies, acne scars, moderate to severe, distensible facial acne scars on the cheek, age-related volume loss, facial lipoatrophy or fat wasting in HIV+ patients, improvement of the appearance of moderate to severe convexity or fullness associated with submental adipose tissue, or fat. In another embodiment, the disease or condition is lip augmentation. In another embodiment the disease or condition is perioral rhytids. In another embodiment, the disease or condition is moderate to severe facial folds and wrinkles such as nasolabial folds. In another embodiment, the disease or condition is moderate to severe facial wrinkles such as smile lines or marionette lines. In another embodiment, the disease or condition is age-related midface contour deficiencies. In another embodiment, the disease or condition is dorsal hand to correct volume deficit. In another embodiment the disease or condition is glabellar lines. In another embodiment, the disease or condition is correction of facila depressions, either due to injury or age-related. In another embodiment the disease or condition is perioral wrinkles. In another embodiment, the disease or condition is lip commissures. In another embodiment, the disease or condition is crow's feet. In another embodiment the disease or condition is forehead wrinkles. In another embodiment, the disease or condition is soft tissue contour deficiencies. In another, the disease or condition is acne scars. In another embodiment, the disease or condition is moderate to severe, distensible facial acne scars on the cheek. In another embodiment, the disease or condition is age-related volume loss. In another embodiment, the disease or condition is facial lipoatrophy or fat wasting in HIV+ patients. In another embodiment, the disease or condition is improvement of the appearance of moderate to severe convexity or fullness associated with submental adipose tissue, or fat.

Facial lipoatrophy is the loss of adipose, or fat, tissue under the skin. Facial lipoatrophy can be directly caused by some anti-retroviral drugs, including those indicated for treatment of Human Immunodeficiency Virus (HIV). Facial lipoatrophy is also caused by an HIV-infection, genetics, having high triglycerides, and/or the natural aging process. The efficacy of treatment regime in a subject suffering from facial lipoatrophy can be measured by methods known to those of ordinary skill in the art, including using a 4-point scale grading system, where a small, localized area of fat loss characterizes a Grade 1, Grade 2 is an area larger than the small, localized area of Grade 1 is affected (generally including jowl or oral commissure), Grade 3 is an area larger than Grade 2 including shadowing within the area, and Grade 4 displays severe fat depletion with muscle and/or bone protrusion.

Acne scarring can occur in subjects suffering from acne. The efficacy of a treatment regimen in a subject suffering from acne scarring can be measured by methods known to those of ordinary skill in the art, including the Self-assessment of Clinical Acne-Related Scars (SCARS) and the Facial Acne Scar Quality of Life (FASQoL) tools.

Facial rhytids is a condition in subjects that is associated with moderate to severe glabellar lines associated with corrugator and/or procerus muscle activity, moderate to severe lateral canthal lines associated with orbicularis oculi activity, and/or moderate to severe forehead lines associated with frontalis muscle activity. The efficacy of a treatment regimen in a subject having facial rhytids can be measured by methods known to those of ordinary skill in the art, including a 4-point Facial Wrinkle Scale (FWS; 0=none, 1=mild, 2=moderate, 3=severe).

Submental fullness is an aesthetic condition where there is an excess of subcutaneous adipose, or fat, tissue in the chin region. The excess subcutaneous adipose tissue under the chin region creates a moderate to severe convexity, commonly known as a “double chin”. The efficacy of a treatment regimen in a subject having submental fullness is measured by 5-point grading scale, where 0=none, 1=minimal, 2=moderate, 3=severe, and 4=extreme. Further assessment of treatment regimen efficacy was performed by magnetic resonance imaging of submental volume for before and after treatment.

Treatment of wrinkles or facial rhytids with Spongilla and dermal fillers assessed by Global Aesthetic Improvement Scale (GAIS) scale ratings. The five-point GAIS scale for aesthetic improvements has five levels: very much improved, much improved, improved, no change, and worse. Data measured by standardized photo documentation.

Treatment of facial or neck deformities, including those with volume loss, with Spongilla and dermal fillers assessed by Merz Aesthetic Scale (MAS) ratings. The five-point MAS scale ranges from 0, no sagging, 1, mild sagging, 2, moderate sagging, 3, severe sagging, to 4, very severe sagging. The five-point Merz scale also applies to wrinkles, ranging from 0, no wrinkles, 1, mild wrinkles, 2, moderate wrinkles, 3, severe wrinkles, and 4, very severe wrinkles. Data measured by standardized photo documentation

In an aspect, provided herein are methods for enhancing skin permeation of a topically applied dermal filler composition in a skin of a subject, comprising applying to the skin a first composition comprising Spongilla, followed by applying a second composition comprising an effective amount of a dermal filler.

In embodiments, the first composition includes Spongilla as described above.

In embodiments, the second composition includes a dermal filler as described above.

In embodiments, the second composition includes deoxycholic acid as described above.

In embodiments, administering includes the first composition being applied to the skin of the subject in the form of a paste. In embodiments, the paste includes water, saline or hydrogen peroxide. In embodiments, the paste is prepared by mixing a powder comprising Spongilla and water, saline, or hydrogen peroxide.

In embodiments, administering includes the first composition being applied to the skin of the subject in the form of a gel. In embodiments, the gel includes water, saline or hydrogen peroxide. In embodiments, the gel contains Spongilla and water, saline, or hydrogen peroxide.

In embodiments, applying the first composition includes massaging the paste or gel into a region of the skin of a subject. In embodiments, the region is selected from a region of the face, lips, nose, under-eye region, eye brow, head, chin, neck, ear, ear lobes, décolletage, elbow, hand, foot, and knee. In embodiments, the region is a region of the face. In embodiments, the region is a region of the lips. In embodiments, the region is a region of the nose. In embodiments, the region is a region of the under-eye. In embodiments, the region is a region of the eye brow. In embodiments, the region is a region of the head. In embodiments, the region is a region of the chin. In embodiments, the region is a region of the neck. In embodiments, the region is a region of the ear. In embodiments, the region is a region of the ear lobes. In embodiments, the region is a region of the décolletage. In embodiments, the region is a region of the elbow. In embodiments, the region is a region of the hand. In embodiments, the region is a region of the foot. In embodiments, the region is a region of the knee.

In another embodiment is provided any of the methods, compositions for use as a medicament, dermal fillers and kits disclosed herein, where the first composition is applied to the skin of the subject prior to the second composition being applied to the skin of the subject. In another embodiment is provided any of the methods, compositions for use as a medicament, dermal fillers and kits disclosed herein, where the first composition is applied to the skin of the subject prior to the second composition being applied to the same area of the skin of the subject. In another embodiment is provided any of the methods, compositions for use as a medicament, dermal fillers and kits disclosed herein, where the first composition is applied to the skin of the subject and is permitted to dry on the skin of the subject prior to application of the second composition to the skin of the subject. In another embodiment is provided any of the methods, compositions for use as a medicament, dermal fillers and kits disclosed herein, where the first composition is applied to the skin of the subject in the form of an aqueous paste, wherein the aqueous component may be water, saline or hydrogen peroxide. In another embodiment is provided any of the methods, compositions for use as a medicament, dermal fillers and kits disclosed herein, where the first composition is applied to the skin of the subject in the form of a gel, wherein the aqueous component may be water, saline or hydrogen peroxide.

In another aspect is provided any of the methods, compositions for use as a medicament, dermal fillers and kits disclosed herein, wherein the first composition is applied to the skin of the subject in the form of an aqueous paste, wherein the aqueous portion may be derived from water or saline. In another aspect is provided any of the methods, compositions for use as a medicament, dermal fillers and kits disclosed herein, wherein the aqueous paste further comprises hydrogen peroxide. In another aspect is provided any of the methods, compositions for use as a medicament, dermal fillers and kits disclosed herein, wherein an aqueous solution of hydrogen peroxide is applied to the skin of the subject following application of the first composition to the skin of the subject. In another aspect is provided any of the methods, compositions for use as a medicament, dermal fillers and kits disclosed herein, wherein the hydrogen peroxide is an aqueous solution. In another aspect is provided any of the methods, compositions for use as a medicament, dermal fillers and kits disclosed herein, wherein the aqueous solution comprises hydrogen peroxide at a w/w concentration of about 3%. In another aspect is provided any of the methods, compositions for use as a medicament, dermal fillers and kits disclosed herein, wherein the first composition is permitted to dry on the skin of the subject.

In another embodiment is provided any of the methods, compositions for use as a medicament, dermal fillers and kits disclosed herein, where the subject applies the second composition including a derm filler to the skin no more than once every 3 months.

In another aspect is provided any of the methods disclosed herein, wherein the first composition comprising Spongilla is applied to the skin of the subject at least once per week for at least one week. In another aspect is provided any of the methods disclosed herein, wherein the first composition comprising Spongilla is applied to the skin of the subject at least two times per week for at least one week, at least three times per week for at least one week, at least 4 times per week for at least one week, at least 5 times per week for at least one week, at least 6 times per week for at least one week, or at least 7 times per week for at least one week.

In another embodiment is provided any of the methods, compositions for use as a medicament, dermal fillers and kits disclosed herein, where the first composition including Spongilla is applied to the skin of the subject at least once per week for at least two weeks. In another aspect is provided any of the methods, compositions for use as a medicament, dermal fillers and kits disclosed herein, where the first composition including Spongilla is applied to the skin of the subject at least at once per week for at least three weeks, at least once per week for at least 4 weeks, at least once per week for at least 5 weeks, at least once per week for at least 6 weeks, at least once per week for at least 7 weeks, at least once per week for at least 8 weeks, at least once per week for at least 9 weeks, at least once per week for at least 10 weeks, at least once per week for at least 11 weeks, at least once per week for at least 12 weeks, at least once per week for at least 13 weeks, at least once per week for at least 14 weeks, at least once per week for at least 15 weeks, at least once per week for at least 16 weeks, at least once per week for at least 17 weeks, at least once per week for at least 18 weeks, at least once per week for at least 19 weeks, at least once per week for at least 20 weeks, at least once per week for at least 21 weeks, at least once per week for at least 22 weeks, at least once per week for at least 23 weeks, at least once per week for at least 24 weeks.

In another embodiment is provided any of the methods, compositions for use as a medicament, dermal fillers and kits disclosed herein, where the first composition including Spongilla is applied to the skin of the subject once per week for 24 weeks, and the second composition including one or more dermal fillers is applied to the skin of the subject only during the first week of treatment. In another aspect is provided any of the methods, compositions for use as a medicament, dermal fillers and kits disclosed herein, where the first composition including Spongilla is applied to the skin of the subject once per week for 20 weeks, and the second composition including one or more dermal fillers is applied to the skin of the subject only during the first week of treatment. In another aspect is provided any of the methods, compositions for use as a medicament, dermal fillers and kits disclosed herein, where the first composition including Spongilla is applied to the skin of the subject once per week for 16 weeks, and the second composition including one or more dermal fillers is applied to the skin of the subject only during the first week of treatment. In another aspect is provided any of the methods, compositions for use as a medicament, dermal fillers and kits disclosed herein, where the first composition including Spongilla is applied to the skin of the subject once per week for 12 weeks, and the second composition including one or more dermal fillers is applied to the skin of the subject only during the first week of treatment. In another aspect is provided any of the methods, compositions for use as a medicament, dermal fillers and kits disclosed herein, where the first composition including Spongilla is applied to the skin of the subject once per week for 8 weeks, and the second composition including one or more dermal fillers is applied to the skin of the subject only during the first week of treatment. In another aspect is provided any of the methods, compositions for use as a medicament, dermal fillers and kits disclosed herein, where the first composition including Spongilla is applied to the skin of the subject once per week for 6 weeks, and the second composition including one or more dermal fillers is applied to the skin of the subject only during the first week of treatment. In another aspect is provided any of the methods, compositions for use as a medicament, dermal fillers and kits disclosed herein, where the first composition including Spongilla is applied to the skin of the subject once per week for 4 weeks, and the second composition including one or more dermal fillers is applied to the skin of the subject only during the first week of treatment.

In another embodiment is provided any of the methods, compositions for use as a medicament, dermal fillers and kits disclosed herein, where the first composition including Spongilla and the second composition including one or more dermal fillers are applied to the skin of the subject in combination once per week, once every two weeks, once every three weeks, once every four weeks, once every five weeks, once every six weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 9 months, once every 12 months, once every 15 months, once every 18 months, once every 21 months, or once every 24 months. In another embodiment, is provided any of the methods, compositions for use as a medicament, dermal fillers and kits disclosed herein, where the first composition including Spongilla and the second composition including one or more dermal fillers are applied to the skin of the subject in combination once every 3 months, once every 6 months, once every 9 months, once every 12 months, once every 15 months, once every 18 months, once every 21 months, or once every 24 months. In another embodiment is provided any of the methods, compositions for use as a medicament, dermal fillers and kits disclosed herein, where the first composition including Spongilla and the second composition including one or more dermal fillers are applied to the skin of the subject in combination once per week. In another embodiment is provided any of the methods, compositions for use as a medicament, dermal fillers and kits disclosed herein, where the first composition including Spongilla and the second composition including one or more dermal fillers are applied to the skin of the subject in combination once every two weeks. In another embodiment is provided any of the methods, compositions for use as a medicament, dermal fillers and kits disclosed herein, where the first composition including Spongilla and the second composition including one or more dermal fillers are applied to the skin of the subject in combination once every three weeks. In another embodiment is provided any of the methods, compositions for use as a medicament, dermal fillers and kits disclosed herein, where the first composition including Spongilla and the second composition including one or more dermal fillers are applied to the skin of the subject in combination once every four weeks. In another embodiment is provided any of the methods, compositions for use as a medicament, dermal fillers and kits disclosed herein, where the first composition including Spongilla and the second composition including one or more dermal fillers are applied to the skin of the subject in combination once every five weeks. In another embodiment is provided any of the methods, compositions for use as a medicament, dermal fillers and kits disclosed herein, where the first composition including Spongilla and the second composition including one or more dermal fillers are applied to the skin of the subject in combination once every six weeks. In another embodiment is provided any of the methods, compositions for use as a medicament, dermal fillers and kits disclosed herein, where the first composition including Spongilla and the second composition including one or more dermal fillers are applied to the skin of the subject in combination once every 7 weeks. In another embodiment is provided any of the methods, compositions for use as a medicament, dermal fillers and kits disclosed herein, where the first composition including Spongilla and the second composition including one or more dermal fillers are applied to the skin of the subject in combination once every 8 weeks. In another embodiment is provided any of the methods, compositions for use as a medicament, dermal fillers and kits disclosed herein, where the first composition including Spongilla and the second composition including one or more dermal fillers are applied to the skin of the subject in combination once every 9 weeks. In another embodiment is provided any of the methods, compositions for use as a medicament, dermal fillers and kits disclosed herein, where the first composition including Spongilla and the second composition including one or more dermal fillers are applied to the skin of the subject in combination once every 10 weeks. In another embodiment is provided any of the methods, compositions for use as a medicament, dermal fillers and kits disclosed herein, where the first composition including Spongilla and the second composition including one or more dermal fillers are applied to the skin of the subject in combination once every 11 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 12 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 13 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 14 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 15 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 16 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 17 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 18 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 19 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 20 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 21 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 22 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 23 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 24 weeks. In another embodiment is provided any of the methods, compositions for use as a medicament, dermal fillers and kits disclosed herein, wherein the first composition including Spongilla and the second composition including one or more dermal fillers are applied to the skin of the subject in combination once every 9 months. In another embodiment is provided any of the methods, compositions for use as a medicament, dermal fillers and kits disclosed herein, wherein the first composition including Spongilla and the second composition including one or more dermal fillers are applied to the skin of the subject in combination once every 12 months. In another embodiment is provided any of the methods, compositions for use as a medicament, dermal fillers and kits disclosed herein, wherein the first composition including Spongilla and the second composition including one or more dermal fillers are applied to the skin of the subject in combination once every 15 months. In another embodiment is provided any of the methods, compositions for use as a medicament, dermal fillers and kits disclosed herein, wherein the first composition including Spongilla and the second composition including one or more dermal fillers are applied to the skin of the subject in combination once every 18 months. In another embodiment is provided any of the methods, compositions for use as a medicament, dermal fillers and kits disclosed herein, wherein the first composition including Spongilla and the second composition including one or more dermal fillers are applied to the skin of the subject in combination once every 21 months. In another embodiment is provided any of the methods, compositions for use as a medicament, dermal fillers and kits disclosed herein, wherein the first composition including Spongilla and the second composition including one or more dermal fillers are applied to the skin of the subject in combination once every 24 months.

In another embodiment is provided any of the methods, compositions for use as a medicament, dermal fillers and kits disclosed herein, where the skin of the subject is cleaned using a non-comedogenic cleanser, water, or a combination of a non-comedogenic cleanser and water following application of the first composition comprising Spongilla. In another embodiment is provided any of the methods, compositions for use as a medicament, dermal fillers and kits disclosed herein, where the skin of the subject is cleaned using a non-comedogenic cleanser, water, or a combination of a non-comedogenic cleanser and water following application of the second composition including one or more dermal filler to the skin of the subject. Non-comedogenic cleansers are those formulated not to cause blocked pores in the skin of subjects to which such cleansers are applied.

In another aspect is provided any of the methods, compositions for use as a medicament, drug products and kits disclosed herein, where the subject is a human.

Treatment using the compositions in the embodiments listed herein so far are contraindicated for patients with sensitivity to any/all of the compositions including Spongilla, dermal filler, antiseptic reagents, and/or reconstitution reagents. The patient may have an allergy, and active infection, glabellar necrosis, allergy to lidocaine, allergy or sensitivity to any dermal filler component, allergy or sensitivity to Spongilla, allergy or sensitivity to any anaerobic or aerobic bacterial protein or molecule, is pregnant, or is nursing.

### Kits

In another aspect is provided a kit, including a first composition and a second composition, where the first composition includes Spongilla and the second composition includes one or more dermal fillers. In another embodiment is provided any of the kits described herein, further including instructions for use in treating the first and the second composition in the treatment in a subject having a skin disease condition. In another embodiment is provided any of the kits described herein, where the first composition includes Spongilla in the form of a powder. In another embodiment is provided any of the kits described herein, where the Spongilla is in the form of a powder including particles that are substantially uniform in size. In embodiments, the second composition includes one or more dermal fillers. In another embodiment is provided any of the kits described herein, where the derm filler is selected from a chemical compound, a mixture of chemical compounds, a biological macromolecule, or an extract made from biological materials. In another embodiment is provided any of the kits described herein, where the one or more dermal fillers is a chemical compound. In another embodiment is provided any of the kits described herein, where the one or more dermal fillers is a biological macromolecule. In another embodiment is provided any of the kits described herein, wheremmm the one or more dermal fillers is an extract made from biological materials

In another aspect is provided a kit, including a first composition, a second composition, and a third composition, where the first composition includes Spongilla and the second composition includes one or more dermal fillers, and the third composition includes a reagent for reconstitution of Spongilla. In another embodiment is provided any of the kits described herein, further including instructions for use in treating the first, second, and third composition in the treatment in a subject having a skin disease condition. In another embodiment is provided any of the kits described herein, where the first composition includes Spongilla in the form of a powder. In another embodiment is provided any of the kits described herein, where the Spongilla is in the form of a powder including particles that are substantially uniform in size. In embodiments, the second composition includes one or more dermal fillers. In another embodiment is provided any of the kits described herein, where the derm filler is selected from a chemical compound, a mixture of chemical compounds, a biological macromolecule, or an extract made from biological materials. In another embodiment is provided any of the kits described herein, where the one or more dermal fillers is a chemical compound. In another embodiment is provided any of the kits described herein, where the one or more dermal fillers is a biological macromolecule. In another embodiment is provided any of the kits described herein, wherein the one or more dermal fillers is an extract made from biological materials. In another embodiment is provided any of the kits described herein, wherein the one or more reconstitution reagents is saline, or buffered saline. In another embodiment is provided any of the kits described herein, wherein the one or more reconstitution reagents is a hydrogel.

In another aspect is provided a kit, including a first composition, a second composition, and a third composition, where the first composition includes Spongilla and the second composition includes one or more dermal fillers, and the third composition includes a antiseptic reagent. In another embodiment is provided any of the kits described herein, further including instructions for use in treating the first, second, and third composition in the treatment in a subject having a skin disease condition. In another embodiment is provided any of the kits described herein, where the first composition includes Spongilla in the form of a powder. In another embodiment is provided any of the kits described herein, where the Spongilla is in the form of a powder including particles that are substantially uniform in size.

In embodiments, the second composition includes one or more dermal fillers. In another embodiment is provided any of the kits described herein, where the derm filler is selected from a chemical compound, a mixture of chemical compounds, a biological macromolecule, or an extract made from biological materials. In another embodiment is provided any of the kits described herein, where the one or more dermal fillers is a chemical compound. In another embodiment is provided any of the kits described herein, where the one or more dermal fillers is a biological macromolecule. In another embodiment is provided any of the kits described herein, wherein the one or more dermal fillers is an extract made from biological materials. In another embodiment is provided any of the kits described herein, wherein the one or more antiseptic reagents is a hydrogen peroxide solution. In another embodiment is provided any of the kits described herein, wherein the one or more antiseptic reagents is a hydrogen peroxide-containing hydrogel. In another embodiment is provided any of the kits described herein, wherein the one or more antiseptic reagents is a non-comedogenic cleanser. In another embodiment is provided any of the kits described herein, wherein the one or more antiseptic reagents is a solution of isopropyl alcohol. In another embodiment is provided any of the kits described herein, wherein the one or more antiseptic reagents is any acceptable skin antiseptic agent commonly used in surgery, including, but not limited to, chlorhexidine, chlorhexidine-gluconate, iodine, hexachlorophine, benzalkonium chloride, and any reconstitutions thereof.

In another aspect is provided a kit, including a first composition, a second composition, a third composition, and a fourth composition, where the first composition includes Spongilla and the second composition includes one or more dermal fillers, the third composition includes a reagent for reconstitution of Spongilla, and the fourth composition includes an antiseptic reagent. In another embodiment is provided any of the kits described herein, further including instructions for use in treating the first, second, third, and fourth composition in the treatment in a subject having a skin disease condition. In another embodiment is provided any of the kits described herein, where the first composition includes Spongilla in the form of a powder. In another embodiment is provided any of the kits described herein, where the Spongilla is in the form of a powder including particles that are substantially uniform in size. In embodiments, the second composition includes one or more dermal fillers. In another embodiment is provided any of the kits described herein, where the derm filler is selected from a chemical compound, a mixture of chemical compounds, a biological macromolecule, or an extract made from biological materials. In another embodiment is provided any of the kits described herein, where the one or more dermal fillers is a chemical compound. In another embodiment is provided any of the kits described herein, where the one or more dermal fillers is a biological macromolecule. In another embodiment is provided any of the kits described herein, wherein the one or more dermal fillers is an extract made from biological materials. In another embodiment is provided any of the kits described herein, wherein the one or more reconstitution reagents is saline, or buffered saline. In another embodiment is provided any of the kits described herein, wherein the one or more reconstitution reagents is a hydrogel. In another embodiment is provided any of the kits described herein, wherein the one or more antiseptic reagents is a hydrogen peroxide solution. In another embodiment is provided any of the kits described herein, wherein the one or more antiseptic reagents is a hydrogen peroxide-containing hydrogel. In another embodiment is provided any of the kits described herein, wherein the one or more antiseptic reagents is a non-comedogenic cleanser. In another embodiment is provided any of the kits described herein, wherein the one or more antiseptic reagents is a solution of isopropyl alcohol. In another embodiment is provided any of the kits described herein, wherein the one or more antiseptic reagents is any acceptable skin antiseptic agent commonly used in surgery, including, but not limited to, chlorhexidine, chlorhexidine-gluconate, iodine, hexachlorophine, benzalkonium.

In another embodiment is provided any of the kits described herein, wherein not less than 50% of the particles comprising the Spongilla powder pass through a US 70-mesh screen.

In another embodiment is provided any of the kits described herein, wherein not less than about 50% of the particles comprising the Spongilla powder pass through a US 70-mesh screen. In another embodiment are methods, wherein not less than about 60%, or about 70%, or about 75%, or about 80%, or about 85%, or about 90%, or about 95%, or about 96%, or about 97%, or about 98%, or about 99% of the particles comprising the Spongilla powder pass through a US 70-mesh screen. In another embodiment is provided any of the kits disclosed herein, wherein not less than about 95%, or about 96%, or about 97%, or about 98%, or about 99% of the particles comprising the Spongilla powder pass through a US 70-mesh screen. In another embodiment is provided any of the kits disclosed herein, wherein not less than about 95% of the particles comprising the Spongilla powder pass through a US 70-mesh screen. In another embodiment is provided any of the kits disclosed herein, wherein not less than about 96% of the particles comprising the Spongilla powder pass through a US 70-mesh screen. In another embodiment is provided any of the kits disclosed herein, wherein not less than about 97% of the particles comprising the Spongilla powder pass through a US 70-mesh screen. In another embodiment is provided any of the kits disclosed herein, wherein not less than about 98% of the particles comprising the Spongilla powder pass through a US 70-mesh screen. In another embodiment is provided any of the kits disclosed herein, wherein not less than about 99% of the particles comprising the Spongilla powder pass through a US 70-mesh screen.

In another embodiment is provided any of the kits disclosed herein, wherein the particles comprising the Spongilla powder have an average length of from about 50 μm to about 500 μm. In another embodiment is provided any of the kits disclosed herein, wherein the particles comprising the Spongilla powder have an average length of from about 50 μm to about 400 μm, or from about 50 μm to about 350 μm, or from about 50 μm to about 300 μm, or from about 50 μm to about 250 μm, or from about 50 μm to about 200 μm, or from about 75 μm to about 500 μm, or from about 75 μm to about 450 μm, or from about 80 μm to about 450 μm, or from about 80 μm to about 400 μm, or from about 85 μm to about 450 μm, or from about 85 μm to about 400 μm, or from about 90 μm to about 450 μm, or from about 90 μm to about 400 μm, or from about 90 μm to about 350 μm, or from about 100 μm to about 450 μm, or from about 100 μm to about 400 μm, or from about 100 μm to about 350 μm, or from about 100 μm to about 300 μm, or from about 100 μm to about 250 μm, or from about 100 μm to about 200 μm, or from about 150 μm to about 500 μm, or from about 100 μm to about 450 μm, or from about 150 μm to about 400 μm, or from about 150 μm to about 350 μm, or from about 150 μm to about 350 μm, or from about 150 μm to about 300 μm, or from about 150 μm to about 250 μm, or from about 150 μm to about 200 μm, or from about 175 μm to about 450 μm, or from about 175 μm to about 400 μm, or from about 175 μm to about 350 μm, or from about 175 μm to about 300 μm, or from about 175 μm to about 250 μm, or from about 175 μm to about 200 μm. In another embodiment is provided any of the kits disclosed herein, wherein the particles comprising the Spongilla powder have an average length of about 50 μm, or about 75 μm, or about 80 μm, or about 85 μm, or about 90 μm, or about 100 μm, or about 125 μm, or about 150 μm, or about 175 μm, or about 200 μm, or about 225 μm, or about 250 μm, or about 300 μm, or about 350 μm, or about 400 μm, or about 450 μm, or about 500 μm. In another embodiment is provided any of the kits disclosed herein, wherein the particles comprising the Spongilla powder have an average length of about 200 μm.

In another embodiment is provided any of the kits disclosed herein, wherein the particles comprising the Spongilla powder have an average diameter of from about 5 μm to about 50 μm. In another embodiment is provided any of the kits disclosed herein, wherein the particles comprising the Spongilla powder have an average diameter of from about 5 μm to about 45 μm, or from about 5 μm to about 40 μm, from about 5 μm to about 35 μm, from about 5 μm to about 30 μm, from about 5 μm to about 25 μm, from about 5 μm to about 20 μm, from about 10 μm to about 45 μm, from about 10 μm to about 40 μm, from about 10 μm to about 35 μm, from about 10 μm to about 30 μm, from about 10 μm to about 25 μm, from about 10 μm to about 20 μm. In another embodiment is provided any of the kits disclosed herein, wherein the particles comprising the Spongilla powder have an average diameter of about 5 μm, or about 10 μm, or about 15 μm, or about 20 μm, or about 25 μm, or about 30 μm, or about 35 μm, or about 40 μm, or about 45 μm, or about 50 μm.

In another embodiment is provided any of the kits disclosed herein, wherein the particles comprising the Spongilla powder have an embodiment ratio of from about 1 to about 100. In another embodiment is provided any of the kits disclosed herein, wherein the particles comprising the Spongilla powder have an embodiment ratio of from about 1 to about 75, or from about 1 to about 50, or from about 1 to about 25, or from about 1 to about 20, or from about 1 to about 15, or from about 5 to about 100, or from about 5 to about 75, or from about 5 to about 50, or from about 5 to about 40, or from about 5 to about 35, or from about 5 to about 30, or from about 5 to about 25, or from about 5 to about 20, or from about 5 to about 15, or from about 7 to about 50, or from about 7 to about 45, or from about 7 to about 40, or from about 7 to about 35, or from about 7 to about 30, or from about 7 to about 25, or from about 10 to about 50, or from about 10 to about 45, or from about 10 to about 40, or from about 10 to about 35, or from about 10 to about 30, or from about 10 to about 25, or from about 10 to about 15. In another embodiment is provided any of the kits disclosed herein, wherein the particles comprising the Spongilla powder have an embodiment ratio of about 5, or about 6, or about 7, or about 8, or about 9, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 35, or about 40, or about 45, or about 50, or about 75, or about 100.

In another embodiment is provided any of the kits disclosed herein, wherein the first composition has a residual moisture content of not more than about 20%. In another embodiment is provided any of the kits disclosed herein, wherein the first composition has a residual moisture content of not more than about 15%, or not more than about 10%, or not more than about 9%, or not more than about 8%, or not more than about 7%, or not more than about 6%, or not more than about 5%, or not more than about 4%, or not more than about 3%, or not more than about 2%, or not more than 1%. In another embodiment is provided any of the kits disclosed herein, wherein the first composition has a residual moisture content of not more than about 5%. In another embodiment is provided any of the kits disclosed herein, wherein the first composition has a residual moisture content of not more than about 4%. In another embodiment is provided any of the kits disclosed herein, wherein the first composition has a residual moisture content of not more than about 3%. In another embodiment is provided any of the kits disclosed herein, wherein the first composition has a residual moisture content of not more than about 2%. In another embodiment is provided any of the kits disclosed herein, wherein the first composition has a residual moisture content of not more than about 1%.

In another embodiment is provided any of the kits disclosed herein, wherein the first composition has a combined aerobic and anaerobic microbial content of not more than about 25×10⁴ colony-forming units per gram (CFU/g). In another embodiment is provided any of the kits disclosed herein, wherein the first composition has a combined aerobic and anaerobic microbial content of not more than about 10×10⁴ CFU/g, or not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another embodiment is provided any of the kits disclosed herein, wherein the first composition has a combined aerobic and anaerobic microbial content of not more than about 1,000 CFU/g. In another embodiment is provided any of the kits disclosed herein, wherein the first composition has a combined aerobic and anaerobic microbial content of not more than about 750 CFU/g. In another embodiment is provided any of the kits disclosed herein, wherein the first composition has a combined aerobic and anaerobic microbial content of not more than about 500 CFU/g. In another embodiment is provided any of the kits disclosed herein, wherein the first composition has a combined aerobic and anaerobic microbial content of not more than about 250 CFU/g. In another embodiment is provided any of the kits disclosed herein, wherein the first composition has a combined aerobic and anaerobic microbial content of not more than about 200 CFU/g. In another embodiment is provided any of the kits disclosed herein, wherein the first composition has a combined aerobic and anaerobic microbial content of not more than about 150 CFU/g. In another embodiment is provided any of the kits disclosed herein, wherein the first composition has a combined aerobic and anaerobic microbial content of not more than about 100 CFU/g. In another embodiment is provided any of the kits disclosed herein, wherein the first composition has a combined aerobic and anaerobic microbial content of not more than about 75 CFU/g. In another embodiment is provided any of the kits disclosed herein, wherein the first composition has a combined aerobic and anaerobic microbial content of not more than about 50 CFU/g. In another embodiment is provided any of the kits disclosed herein, wherein the first composition has a combined aerobic and anaerobic microbial content of not more than about 25 CFU/g. In another embodiment is provided any of the kits disclosed herein, wherein the first composition has a combined aerobic and anaerobic microbial content of not more than about 20 CFU/g. In another embodiment is provided any of the kits disclosed herein, wherein the first composition has a combined aerobic and anaerobic microbial content of not more than about 10 CFU/g. In another embodiment is provided any of the kits disclosed herein, wherein the first composition has a combined aerobic and anaerobic microbial content of not more than about 1 CFU/g.

In another embodiment is provided any of the kits disclosed herein, wherein the first composition has a combined yeast and mold content of not more than about 25×10⁴ colony-forming units per gram (CFU/g). In another embodiment is provided any of the kits disclosed herein, wherein the first composition has a combined yeast and mold content of not more than about or not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another embodiment is provided any of the kits disclosed herein, wherein the first composition has a combined yeast and mold content of not more than about 1,000 CFU/g. In another embodiment is provided any of the kits disclosed herein, wherein the first composition has a combined yeast and mold content of not more than about 750 CFU/g. In another embodiment is provided any of the kits disclosed herein, wherein the first composition has a combined yeast and mold content of not more than about 500 CFU/g. In another embodiment is provided any of the kits disclosed herein, wherein the first composition has a combined yeast and mold content of not more than about 250 CFU/g. In another embodiment is provided any of the kits disclosed herein, wherein the first composition has a combined yeast and mold content of not more than about 200 CFU/g. In another embodiment is provided any of the kits disclosed herein, wherein the first composition has a combined yeast and mold content of not more than about 150 CFU/g. In another embodiment is provided any of the kits disclosed herein, wherein the first composition has a combined yeast and mold content of not more than about 100 CFU/g. In another embodiment is provided any of the kits disclosed herein, wherein the first composition has a combined yeast and mold content of not more than about 75 CFU/g. In another embodiment is provided any of the kits disclosed herein, wherein the first composition has a combined yeast and mold content of not more than about 50 CFU/g. In another embodiment is provided any of the kits disclosed herein, wherein the first composition has a combined yeast and mold content of not more than about 25 CFU/g. In another embodiment is provided any of the kits disclosed herein, wherein the first composition has a combined yeast and mold content of not more than about 20 CFU/g. In another embodiment is provided any of the kits disclosed herein, wherein the first composition has a combined yeast and mold content of not more than about 10 CFU/g. In another embodiment is provided any of the kits disclosed herein, wherein the first composition has a combined yeast and mold content of not more than about 1 CFU/g.

In another embodiment is provided any of the kits disclosed herein, wherein the amount of Coliform bacteria in the first composition is not more than about 25×10⁴ colony-forming units per gram (CFU/g). In another embodiment is provided any of the kits disclosed herein, wherein the amount of Coliform bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another embodiment is provided any of the kits disclosed herein, wherein the first composition has a Coliform bacteria content of not more than about 1,000 CFU/g. In another embodiment is provided any of the kits disclosed herein, wherein the first composition has a Coliform bacteria content of not more than about 750 CFU/g. In another embodiment is provided any of the kits disclosed herein, wherein the first composition has a Coliform bacteria content of not more than about 500 CFU/g. In another embodiment is provided any of the kits disclosed herein, wherein the first composition has a Coliform bacteria content of not more than about 250 CFU/g. In another embodiment is provided any of the kits disclosed herein, wherein the first composition has a Coliform bacteria content of not more than about 200 CFU/g. In another embodiment is provided any of the kits disclosed herein, wherein the first composition has a Coliform bacteria content of not more than about 150 CFU/g. In another embodiment is provided any of the kits disclosed herein, wherein the first composition has a Coliform bacteria content of not more than about 100 CFU/g. In another embodiment is provided any of the kits disclosed herein, wherein the first composition has a Coliform bacteria content of not more than about 75 CFU/g. In another embodiment is provided any of the kits disclosed herein, wherein the first composition has a Coliform bacteria content of not more than about 50 CFU/g. In another embodiment is provided any of the kits disclosed herein, wherein the first composition has a Coliform bacteria content of not more than about 25 CFU/g. In another embodiment is provided any of the kits disclosed herein, wherein the first composition has a Coliform bacteria content of not more than about 20 CFU/g. In another embodiment is provided any of the kits disclosed herein, wherein the first composition has a Coliform bacteria content of not more than about 10 CFU/g. In another embodiment is provided any of the kits disclosed herein, wherein the first composition has a Coliform bacteria content of not more than about 1 CFU/g. In another embodiment is provided any of the kits disclosed herein, wherein the first composition has no detectable Coliform bacterial content.

In another embodiment is provided any of the kits disclosed herein, wherein the amount of Salmonella in the first composition is not more than about 25×10⁴ colony-forming units per gram (CFU/g). In another embodiment is provided any of the kits disclosed herein, wherein the amount of Salmonella in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another embodiment is provided any of the kits disclosed herein, wherein the first composition has a Salmonella content of not more than about 1,000 CFU/g. In another embodiment is provided any of the kits disclosed herein, wherein the first composition has a Salmonella content of not more than about 750 CFU/g. In another embodiment is provided any of the kits disclosed herein, wherein the first composition has a Salmonella content of not more than about 500 CFU/g. In another embodiment is provided any of the kits disclosed herein, wherein the first composition has a Salmonella content of not more than about 250 CFU/g. In another embodiment is provided any of the kits disclosed herein, wherein the first composition has a Salmonella content of not more than about 200 CFU/g. In another embodiment is provided any of the kits disclosed herein, wherein the first composition has a Salmonella content of not more than about 150 CFU/g. In another embodiment is provided any of the kits disclosed herein, wherein the first composition has a Salmonella content of not more than about 100 CFU/g. In another embodiment is provided any of the kits disclosed herein, wherein the first composition has a Salmonella content of not more than about 75 CFU/g. In another embodiment is provided any of the kits disclosed herein, wherein the first composition has a Salmonella content of not more than about 50 CFU/g. In another embodiment is provided any of the kits disclosed herein, wherein the first composition has a Salmonella content of not more than about 25 CFU/g. In another embodiment is provided any of the kits disclosed herein, wherein the first composition has a Salmonella content of not more than about 20 CFU/g. In another embodiment is provided any of the kits disclosed herein, wherein the first composition has a Salmonella content of not more than about 10 CFU/g. In another embodiment is provided any of the kits disclosed herein, wherein the first composition has a Salmonella content of not more than about 1 CFU/g. In another embodiment is provided any of the kits disclosed herein, wherein the first composition has no detectable Salmonella content.

In another embodiment is provided any of the kits disclosed herein, wherein the amount of Pseudomonas aeruginosa bacteria in the first composition is not more than about 25×10⁴ colony-forming units per gram (CFU/g). In another embodiment is provided any of the kits disclosed herein, wherein the amount of Pseudomonas aeruginosa bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another embodiment is provided any of the kits disclosed herein, wherein the first composition has a Pseudomonas aeruginosa bacteria content of not more than about 1,000 CFU/g. In another embodiment is provided any of the kits disclosed herein, wherein the first composition has a Pseudomonas aeruginosa bacteria content of not more than about 750 CFU/g. In another embodiment is provided any of the kits disclosed herein, wherein the first composition has a Pseudomonas aeruginosa bacteria content of not more than about 500 CFU/g. In another embodiment is provided any of the kits disclosed herein, wherein the first composition has a Pseudomonas aeruginosa bacteria content of not more than about 250 CFU/g. In another embodiment is provided any of the kits disclosed herein, wherein the first composition has a Pseudomonas aeruginosa bacteria content of not more than about 200 CFU/g. In another embodiment is provided any of the kits disclosed herein, wherein the first composition has a Pseudomonas aeruginosa bacteria content of not more than about 150 CFU/g. In another embodiment is provided any of the kits disclosed herein, wherein the first composition has a Pseudomonas aeruginosa bacteria content of not more than about 100 CFU/g. In another embodiment is provided any of the kits disclosed herein, wherein the first composition has a Pseudomonas aeruginosa bacteria content of not more than about 75 CFU/g. In another embodiment is provided any of the kits disclosed herein, wherein the first composition has a Pseudomonas aeruginosa bacteria content of not more than about 50 CFU/g. In another embodiment is provided any of the kits disclosed herein, wherein the first composition has a Pseudomonas aeruginosa bacteria content of not more than about 25 CFU/g. In another embodiment is provided any of the kits disclosed herein, wherein the first composition has a Pseudomonas aeruginosa bacteria content of not more than about 20 CFU/g. In another embodiment is provided any of the kits disclosed herein, wherein the first composition has a Pseudomonas aeruginosa bacteria content of not more than about 10 CFU/g. In another embodiment is provided any of the kits disclosed herein, wherein the first composition has a Pseudomonas aeruginosa bacteria content of not more than about 1 CFU/g. In another embodiment is provided any of the kits disclosed herein, wherein the first composition has no detectable Pseudomonas aeruginosa bacteria content.

In another embodiment is provided any of the kits disclosed herein, wherein the amount of Staphylococcus aureus bacteria in the first composition is not more than about 25×10⁴ colony-forming units per gram (CFU/g). In another embodiment is provided any of the kits disclosed herein, wherein the amount of Staphylococcus aureus bacteria in the first composition is not more than about 5×10⁴ CFU/g, or not more than about 1×10⁴ CFU/g, or not more than about 5×10³ CFU/g, or not more than about 1×10³ CFU/g, or not more than about 10,000 CFU/g, or not more than about 7,500 CFU/g, or not more than about 5,000 CFU/g, or not more than about 2,500 CFU/g, or not more than about 2,000 CFU/g, or not more than about 1,500 CFU/g, or not more than about 1,000 CFU/g, or not more than about 750 CFU/g, or not more than about 500 CFU/g, or not more than about 250 CFU/g, or not more than about 200 CFU/g, or not more than about 150 CFU/g, or not more than about 100 CFU/g, or not more than about 75 CFU/g, or not more than about 50 CFU/g, or not more than about 25 CFU/g, or not more than about 15 CFU/g, or not more than about 10 CFU/g, or not more than about 5 CFU/g, or not more than about 1 CFU/g. In another embodiment is provided any of the kits disclosed herein, wherein the first composition has a Staphylococcus aureus bacteria content of not more than about 1,000 CFU/g. In another embodiment is provided any of the kits disclosed herein, wherein the first composition has a Staphylococcus aureus bacteria content of not more than about 750 CFU/g. In another embodiment is provided any of the kits disclosed herein, wherein the first composition has a Staphylococcus aureus bacteria content of not more than about 500 CFU/g. In another embodiment is provided any of the kits disclosed herein, wherein the first composition has a Staphylococcus aureus bacteria content of not more than about 250 CFU/g. In another embodiment is provided any of the kits disclosed herein, wherein the first composition has a Staphylococcus aureus bacteria content of not more than about 200 CFU/g. In another embodiment is provided any of the kits disclosed herein, wherein the first composition has a Staphylococcus aureus bacteria content of not more than about 150 CFU/g. In another embodiment is provided any of the kits disclosed herein, wherein the first composition has a Staphylococcus aureus bacteria content of not more than about 100 CFU/g. In another embodiment is provided any of the kits disclosed herein, wherein the first composition has a Staphylococcus aureus bacteria content of not more than about 75 CFU/g. In another embodiment is provided any of the kits disclosed herein, wherein the first composition has a Staphylococcus aureus bacteria content of not more than about 50 CFU/g. In another embodiment is provided any of the kits disclosed herein, wherein the first composition has a Staphylococcus aureus bacteria content of not more than about 25 CFU/g. In another embodiment is provided any of the kits disclosed herein, wherein the first composition has a Staphylococcus aureus bacteria content of not more than about 20 CFU/g. In another embodiment is provided any of the kits disclosed herein, wherein the first composition has a Staphylococcus aureus bacteria content of not more than about 10 CFU/g. In another embodiment is provided any of the kits disclosed herein, wherein the first composition has a Staphylococcus aureus bacteria content of not more than about 1 CFU/g. In another embodiment is provided any of the kits disclosed herein, wherein the first composition has no detectable Staphylococcus aureus bacteria content.

In another embodiment is provided any of the kits disclosed herein, wherein the first composition is packaged prior to use. In another embodiment is provided any of the kits disclosed herein, wherein the first composition is prepared by heating to at least about 70° C. prior to being packaged. In another embodiment is provided any of the kits disclosed herein, wherein the first composition is prepared by heating to at least about 50° C., or at least about 60° C., or at least about 75° C., or at least about 80° C., or at least about 85° C., or at least about 90° C., or at least about 100° C., or at least about 110° C., or at least about 115° C., or at least about 120° C., or at least about 125° C., or at least about 130° C., or at least about 135° C., or at least about 140° C., or at least about 150° C., or at least about 160° C., or at least about 170° C., or at least about 180° C., or at least about 190° C., or at least about 200° C. prior to being packaged.

In another embodiment is provided any of the kits disclosed herein, wherein the first composition is heated to at least about 70° C. for at least about 5 minutes prior being packaged. In another embodiment is provided any of the kits disclosed herein, wherein the first composition is heated to at least about 70° C. for at least about 10 minutes, or at least about 15 minutes, or at least about 20 minutes, or at least about 25 minutes, or at least about 30 minutes, or at least about 35 minutes, or at least about 40 minutes, or at least about 45 minutes, or at least about 50 minutes, or at least about 55 minutes, or at least about 60 minutes, or at least about 75 minutes, or at least about 90 minutes, or at least about 120 minutes, or at least about 180 minutes, or at least about 4 hours, or at least about 5 hours, or at least about 6 hours, or at least about 7 hours, or at least about 8 hours, or at least about 9 hours, or at least about 10 hours, or at least about 11 hours, or at least about 12 hours, or at least about 24 hours prior being packaged.

In another embodiment is provided any of the kits disclosed herein, wherein the first composition is prepared by treatment with ionizing radiation, such as gamma radiation, prior to being packaged or after packaging. For example, gamma irradiation may be performed on the raw Spongilla material prior to grinding to reduce the particle size, following grinding to reduce the particle size, the materials packaged in bulk and or the materials following packaging in unit dose containers. The materials comprising the kits disclosed herein may be treated with ionizing radiation, such as gamma radiation, using methods and equipment known to those of ordinary skill in the art, such as gamma irradiators or electron beam irradiators. In another embodiment is provided any of the kits disclosed herein, wherein the first composition is prepared by treating with ionizing radiation, such as gamma radiation, to deliver an absorbed radiation dose between about 1 kGy and about 50 kGy prior to being packaged. In another embodiment is provided any of the kits disclosed herein, wherein the first composition is prepared by treating with ionizing radiation, such as gamma radiation, to deliver an absorbed radiation dose between about 1 kGy and about 45 kGy, or between about 1 kGy and about 40 kGy, between about 1 kGy and about 35 kGy, between about 1 kGy and about 30 kGy, or between about 1 kGy and about 25 kGy or between about 5 kGy and about 50 kGy, or between about 5 kGy and about 45 kGy, or between about 5 kGy and about 40 kGy, or between about 5 kGy and about 35 kGy, or between about 5 kGy and about 30 kGy, or between about 5 kGy and about 25 kGy, or between about 10 kGy and about 50 kGy, or between about 10 kGy and about 45 kGy, or between about 10 kGy and about 40 kGy, or between about 10 kGy and about 35 kGy, or between about 10 kGy and about 30 kGy, or between about 10 kGy and about 25 kGy, or between about 15 kGy and about 50 kGy, or between about 15 kGy and about 45 kGy, or between about 15 kGy and about 40 kGy, or between about 15 kGy and about 35 kGy, or between about 15 kGy and about 30 kGy, or between about 15 kGy and about 25 kGy. In another embodiment is provided any of the methods disclosed herein, wherein the first composition is prepared by treating with ionizing radiation, such as gamma radiation, to deliver an absorbed radiation dose of about 1 kGy, or about 5 kGy, or about 10 kGy, 11 kGy, or about 12 kGy, or about 13 kGy, or about 14 kGy, or about 15 kGy, or about 16 kGy, or about 17 kGy, or about 18 kGy, or about 19 kGy, or about 20 kGy, or about 21 kGy, or about 22 kGy, or about 23 kGy, or about 24 kGy, or about 25 kGy, or about 26 kGy, or about 27 kGy, or about 28 kGy, or about 29 kGy, or about 30 kGy, or about 31 kGy, or about 32 kGy, or about 33 kGy, or about 34 kGy, or about 35 kGy, or about 36 kGy, or about 37 kGy, or about 38 kGy, or about 39 kGy, or about 40 kGy, or about 41 kGy, or about 42 kGy, or about 43 kGy, or about 44 kGy, or about 45 kGy, or about 46 kGy, or about 47 kGy, or about 48 kGy, or about 49 kGy, or about 50 kGy.

In another embodiment is provided any of the kits disclosed herein, wherein the first composition further comprises an aqueous solution of hydrogen peroxide. In another embodiment is provided any of the kits disclosed herein, wherein the hydrogen peroxide is at a concentration of from about 0.1% w/w to about 50% w/w, or from about 0.1% w/w to about 45% w/w, or from about 0.1% w/w to about 40% w/w, or from about 0.1% w/w to about 35% w/w, or from about 0.1% w/w to about 30% w/w, or from about 0.1% w/w to about 25% w/w, or from about 0.1% w/w to about 20% w/w, or from about 0.1% w/w to about 15% w/w, or from about 0.1% w/w to about 10% w/w, or from about 0.1% w/w to about 9% w/w, or from about 0.1% w/w to about 8% w/w, or from about 0.1% w/w to about 8% w/w, or from about 0.1% w/w to about 7% w/w, or from about 0.1% w/w to about 6% w/w, or from about 0.1% w/w to about 5% w/w, or from about 0.1% w/w to about 4% w/w, or from about 0.1% w/w to about 3% w/w, or from about 0.1% w/w to about 2% w/w, or from about 0.1% w/w to about 1% w/w, or from about 0.5% w/w to about 45% w/w, or from about 1% w/w to about 45% w/w, or from about 1% w/w to about 40% w/w, or from about 1% w/w to about 35% w/w, or from about 1% w/w to about 30% w/w, or from about 1% w/w to about 25% w/w, or from about 1% w/w to about 20% w/w, or from about 1% w/w to about 15% w/w, or from about 1% w/w to about 10% w/w, or from about 1% w/w to about 9% w/w, or from about 1% w/w to about 8% w/w, or from about 1% w/w to about 8% w/w, or from about 1% w/w to about 7% w/w, or from about 1% w/w to about 6% w/w, or from about 1% w/w to about 5% w/w, or from about 1% w/w to about 4% w/w, or from about 1% w/w to about 3% w/w, or from about 1% w/w to about 2% w/w, or from about 2% w/w to about 45% w/w, or from about 2% w/w to about 40% w/w, or from about 2% w/w to about 35% w/w, or from about 2% w/w to about 30% w/w, or from about 2% w/w to about 25% w/w, or from about 2% w/w to about 20% w/w, or from about 2% w/w to about 15% w/w, or from about 2% w/w to about 10% w/w, or from about 2% w/w to about 9% w/w, or from about 2% w/w to about 8% w/w, or from about 1% w/w to about 7% w/w, or from about 2% w/w to about 6% w/w, or from about 2% w/w to about 5% w/w, or from about 2% w/w to about 4% w/w, or from about 2% w/w to about 3% w/w. In another embodiment is provided any of the kits disclosed herein, wherein the hydrogen peroxide is at a concentration of about 0.1% w/w, or about 0.5% w/w, or about 1% w/w, or about 2% w/w, or about 3% w/w, or about 4% w/w, or about 5% w/w, or about 6% w/w, or about 7% w/w, or about 8% w/w, or about 9% w/w, or about 10% w/w, or about 15% w/w, or about 20% w/w, or about 25% w/w, or about 30% w/w, or about 35% w/w, or about 40% w/w, or about 45% w/w, or about 50% w/w. In another embodiment is provided any of the methods disclosed herein, wherein the hydrogen peroxide is at a concentration of about 3% w/w. Aqueous hydrogen peroxide solutions that may be useful in treating skin conditions in a subject as disclosed herein are commercially available or may be prepared by methods known to those of ordinary skill in the art.

In another embodiment is provided any of the kits disclosed herein, further comprising a gel or cream comprising hydrogen peroxide. Such gels or creams are generally commercially available any may contain from about 0.5% w/w to about 50% w/w hydrogen peroxide. For example, a gel containing about 1% w/w, or about 2% w/w, or about 3% w/w, or about 4% w/w, or about 6% w/w, or about 7% w/w, or about 8% w/w, or about 9% w/w, or about 10% w/w, or about 15% w/w, or about 20% w/w, or about 25% w/w, or about 30% w/w, or about 40% w/w, or about 45% w/w, or about 50% w/w hydrogen peroxide may be used in any of the methods and kits disclosed herein in combination with the first composition and the second composition.

In another embodiment is provided any of the kits disclosed herein, wherein the Spongilla is Spongilla lacustris.

The compositions disclosed herein, such as the first composition comprising Spongilla, may further include one or more conventional pharmaceutical carriers or excipients. Suitable pharmaceutical carriers and excipients include inert diluents, binders (such as starches), fillers (such as colloidal silicon dioxide, sugars, including lactose, sucrose, mannitol, or sorbitol; and cellulose preparations, for example, maize starch, wheat starch, rice starch, potato starch, gelatin, gum, methyl cellulose, hydroxypropylmethyl cellulose, sodium carboxymethylcellulose, or polyvinylpyrrolidone (PVP)), bulking agents, lubricants (such as magnesium stearate, sodium lauryl sulfate and talc), coloring matters or dyes and, if desired, emulsifying agents or suspending agents, together with diluents such as water, saline, ethanol, propylene glycol, glycerin, or combinations thereof.

In another embodiment is provided any of the kits disclosed herein, further comprising a dermal filler including polymers, co-polymers, cross-linked polymers, and/or cross-linked copolymers of hyaluranonic acid, calcium hydroxylapatite, polyalkylimide, polymethylmethacrylate, collagens, polymethyl-methacrylate microspheres (PMMA), polylactic acid, polycaprolactone, carboxymethyl cellulose, poly-L-lactic acid. In embodiments, a dermal filler includes polymers, co-polymers, cross-linked polymers, and/or cross-linked copolymers of hyaluranonic acid. In embodiments, a dermal filler includes polymers, co-polymers, cross-linked polymers, and/or cross-linked copolymers of calcium hydroxlyapatite. In embodiments, a dermal filler includes polymers, co-polymers, cross-linked polymers, and/or cross-linked copolymers of polyalkylimide. In embodiments, a dermal filler includes polymers, co-polymers, cross-linked polymers, and/or cross-linked copolymers of polymethylmethacrylate. In embodiments, a dermal filler includes polymers, co-polymers, cross-linked polymers, and/or cross-linked copolymers of collagen. In embodiments, a dermal filler includes polymers, co-polymers, cross-linked polymers, and/or cross-linked copolymers of polymethyl-methacrylate microspheres (PMMA). In embodiments, a dermal filler includes polymers, co-polymers, cross-linked polymers, and/or cross-linked copolymers of polylactic acid. In embodiments, a dermal filler includes polymers, co-polymers, cross-linked polymers, and/or cross-linked copolymers of polycaprolactone. In embodiments, a dermal filler includes polymers, co-polymers, cross-linked polymers, and/or cross-linked copolymers of carboxymethyl cellulose. In embodiments, a dermal filler includes polymers, co-polymers, cross-linked polymers, and/or cross-linked copolymers of poly-L-lactic acid.

In another embodiment is provided any of the kits disclosed herein, further comprising a topical dermal filler, including glycerin, peptides derived from collagen, alguronic acid, low molecular weight hyaluronic acid polymers, sodium aceylated hyaluronate, glycolic acid, lactic acid, L-lactic acid, amino acids, NIA-114™, an acetyl peptide, Activen™ XEP-18, and/or Erasa™ XEP-30. In embodiments, a dermal filler includes glycerin. In embodiments, a dermal filler includes peptides derived from collagen. In embodiments, a dermal filler includes alguronic acid. In embodiments, a dermal filler includes low molecular weight hyaluronic acid polymers. In embodiments, a dermal filler includes sodium aceylated hyaluronate. In embodiments, a dermal filler includes glycolic acid. In embodiments, a dermal filler includes lactic acid. In embodiments, a dermal filler includes L-lactic acid. In embodiments, a dermal filler includes amino acids. In embodiments, a dermal filler includes NIA-114®. In embodiments, a dermal filler includes acetyl peptide. In embodiments, a dermal filler includes Activen™ XEP-18. In embodiments, a dermal filler includes Erasa™ XEP-30.

In another embodiment is provided any of the kits disclosed herein, further a composition comprising deoxycholic acid.

The compositions disclosed herein may be in unit dosage forms suitable for single administration of precise dosages. In another embodiment is provided any of the methods or kits disclosed herein, where the unit dosage forms of the first compositions and/or the second composition are suitable for two administrations, three administrations, four administrations, five administrations, six administrations, seven administrations, eight administrations, 9 administrations, 10 administrations, 11 administrations, 12 administrations, 13 administrations, 14 administrations, 15 administrations, 16 administrations, 17 administrations, 18 administrations, 19 administrations, 20 administrations, 21 administrations, 22 administrations, 23 administrations, 24 administrations, 25 administrations, 26 administrations, 27 administrations, 28 administrations, 29 administrations, 30 administrations, administrations for two months, administrations for three months, administrations for four months, administrations for five months, administrations for six months, administrations for seven months, administrations for eight months, administrations for nine months, administrations for ten months, administrations for eleven months, or administrations for 12 months.

It will be appreciated that the actual dosages of the compositions disclosed herein, may vary according to the composition being used, the mode of administration, and the particular site of the subject being treated, and the skin condition being treated. Those skilled in the art using conventional dosage-determination tests in view of the experimental data for a given composition may ascertain optimal dosages for a given set of conditions. This amount will vary depending upon a variety of factors, including but not limited to the characteristics of the compositions and formulations disclosed herein (including activity, pharmacokinetics, pharmacodynamics, and bioavailability thereof), the physiological condition of the subject treated (including age, sex, disease type and stage, general physical condition, responsiveness to a given dosage, and type of medication) or cells, the nature of the pharmaceutically acceptable carrier mg/kg or carriers in the formulation, and the route of administration. Further, an effective or therapeutically effective amount may vary depending on whether the one or more compositions and formulations disclosed herein is administered alone or in combination with other derm filler(s), other therapy/therapies or other therapeutic method(s) or modality/modalities. One skilled in the clinical and pharmacological arts will be able to determine an effective amount or therapeutically effective amount through routine experimentation, namely by monitoring a cell's or subject's response to administration of the one or more compositions and formulations disclosed herein and adjusting the dosage accordingly.

Dosage regimens using the first composition and the second composition may be adjusted to provide the optimum desired response. Dosage unit form, as used herein, refers to physically discrete units suited as unitary dosages for the subjects to be treated; each unit containing a predetermined quantity of the compositions disclosed herein, calculated to produce the desired therapeutic effect in association with the required pharmaceutical carrier. The specification for the dosage unit forms of the compositions disclosed herein are dictated by and directly dependent on (a) the characteristics of the composition and the particular therapeutic or prophylactic effect to be achieved, and (b) the limitations inherent in the art of compounding such a composition for the treatment a particular condition in a subject.

Thus, the skilled artisan would appreciate, based upon the disclosure provided herein, that the dose and dosing regimen using the compositions disclosed herein may be adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the presently disclosed methods.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. The embodiments disclosed herein are intended to encompass intra-subject dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

In some embodiments, the compositions may be used in combination with one or more additional compositions useful in treating skin conditions in a subject which are described above. When a combination therapy is used, the one or more additional compositions may be administered sequentially or simultaneously with the first composition and/or the second composition disclosed herein. In some embodiments, the additional compositions is administered to a subject prior to, at the same time as, or following administration of the first composition and/or the second composition disclosed herein. In some embodiments, the additional composition is administered to the subject prior to the administration of the first composition and/or the second composition disclosed herein. In some embodiments, the additional composition is administered to the subject at the same time the first composition and/or the second composition disclosed herein are administered to the subject. In some embodiments, the additional composition is administered to the subject following to the administration of the first composition and/or the second composition disclosed herein. Among the additional compositions that may be used according to any of the methods disclosed herein include, but are not limited to, cromolyn sodium (also known as sodium cromoglycate), topical alpha agonists (including, but not limited to, oxymetazoline hydrochloride, clonidine hydrochloride, apraclonidine hydrochloride, and brimonidine tartrate), topical antibiotics (including, but not limited to, tetracyclines [tetracycline, doxycycline, minocycline, sarecycline], clindamycin, and erythromycin), benzoyl peroxide, salicylic acid, azelaic acid, retinoids, topical anticholinergics (including, but not limited to, oxybutynin, glycopyrrolate, propantheline), topical prostaglandin analogs (including, but not limited to, latanoprost, bimatoprost, travoprost, and tafluprost), and topical hydroquinone or a combination of fluocinolone acetonide, hydroquinone, and tretinoin (sold as Tri-Luma® cream).

As will be understood by one skilled in the art, for all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (i) etc., are presented merely for ease of reading the specification and claims. The use of headings in the specification or claims does not require the steps or elements be performed in alphabetical or numerical order or the order in which they are presented.

The preparations and examples of a number of embodiments disclosed herein are intended to be illustrative and not limiting. All starting materials are available commercially or are described in the literature. All temperatures are reported in ° C.

### EXAMPLES

### Example 1: Use of Spongilla and Hyaluronic Acid for the Treatment of Moderate to Severe Facial Wrinkles and Folds, Such as Nasolabial Folds

Subjects 21 years of age and older having moderate to severe facial wrinkles and folds, such as nasolabial folds on the 5-point wrinkle severity rating scale (WSRS), are treated according to the regimen below.

Week 1: The subject's skin is washed and dried with a mild, hypoallergenic cleanser (eg, Cetaphil®). A prefilled syringe containing hyaluronic acid is removed from the refrigerator and allowed to reach room temperature (30 minutes) without removing the needle cap. 6 mL of 3% hydrogen peroxide USP is added to 2 grams of Spongilla powder, the mixture is stirred until all powder has mixed with the hydrogen peroxide. The Spongilla mixture is massaged into the areas of the subject's skin that are affected, taking care to avoid mucous membranes (eg, eyes, mouth, and nostrils), and the Spongilla composition is allowed to dry on the application area. The Spongilla composition is then removed using non-hypoallergenic wipes or water plus a cleanser. The composition comprising from 20 mg of hyaluronic acid is applied to the skin of the subject where Spongilla had been applied taking care to avoid mucous membranes (eg, eyes, mouth, and nostrils), and the hyaluronic acid is massaged into the subject's skin. The hyaluronic acid composition is allowed to remain on the application area with the subject resting comfortably. After 15 minutes, the application area is washed with hypoallergenic wipes or water plus hypoallergenic cleanser. The procedure above is repeated once every six months following the first application.

Thereafter, subjects report an improvement in the facial wrinkles and folds, such as nasolabial folds based on a ≥1-point improvement on the WSRS, including a 100% seeing some improvement (improved, much improved and very much improved) at day fourteen when subjects were asked to rate the level of improvement seen in their nasolabial folds. Subjects may receive touch-up treatments 30 days after the initial treatment using the procedure described above.

### Example 2: Use of Spongilla and Hyaluronic Acid for the Treatment of Moderate to Severe Atrophic, Distensible Facial Acne Scars

Subjects 21 years of age and older having moderate to severe, atrophic, distensible facial acne scars on the cheek using the 4-point acne scar rating scale (ACRS), are treated according to the regimen below.

Week 1: The subject's skin is washed and dried with a mild, hypoallergenic cleanser (eg, Cetaphil®). A prefilled syringe containing non-resorbable polymethylmethacrylate microspheres, 30 to 50 microns in diameter, suspended in a water-based carrier gel composed of 3.5% bovine collagen (PMMA-Collagen) is removed from its packaging without attaching the needle. 6 mL of 3% hydrogen peroxide USP is added to 2 grams of Spongilla powder, the mixture is stirred until all powder has mixed with the hydrogen peroxide. The Spongilla mixture is massaged into the areas of the subject's skin that are affected, taking care to avoid mucous membranes (eg, eyes, mouth, and nostrils), and the Spongilla composition is allowed to dry on the application area. The Spongilla composition is then removed using non-hypoallergenic wipes or water plus a cleanser. The composition comprising PMMA-Collagen is applied to the skin of the subject where Spongilla had been applied taking care to avoid mucous membranes (eg, eyes, mouth, and nostrils), and the PMMA-Collagen is massaged into the subject's skin. The PMMA-Collagen composition is allowed to remain on the application area with the subject resting comfortably. After 15 minutes, the application area is washed with hypoallergenic wipes or water plus hypoallergenic cleanser. The procedure above is repeated once every six months following the first application.

Thereafter, subjects experience an improvement in atrophic, distensible facial acne scars on the cheek based on 50% or more of treated scars improving by 2 or more points at month 6 based on the blinded-investigator assessment.

Subjects may receive touch-up treatment 30 days after the initial treatment using the procedure described above.

1. A composition comprising:
(a) a first composition comprising Spongilla; and
(b) a second composition comprising a dermal filler.

2. The composition of claim 1, wherein the Spongilla is in the form of a powder.

3. The composition of claim 2, wherein the powder comprises particles that are substantially uniform in size.

4. The composition of any one of claims 2-3, wherein not less than 50% of the particles comprising the Spongilla powder pass through a US 70-mesh screen.

5. The composition of any one of claims 2-4, wherein the particles comprising the Spongilla powder have an average length of from about 50 μm to about 500 μm.

6. The composition of any one of claims 2-5, wherein the particles comprising the Spongilla powder have an average diameter of from about 5 μm to about 50 μm.

7. The composition of any one of claims 2-6, wherein the particles comprising the Spongilla powder have an aspect ratio of from about 1 to 100.

8. The composition of any one of claims 1-7, wherein the first composition has a residual moisture content of not more than about 10%.

9. The composition of any one of claims 1-8, wherein the amount of the first composition comprising Spongilla is from about 0.5 grams to about 50 grams.

10. The composition of any one of claims 1-9, wherein the first composition is applied to the skin of the subject in the form of a paste or gel.

11. The composition of claim 10, wherein the paste further comprises water or saline or hydrogen peroxide.

12. The composition of any one of claim 10 or 11, wherein the paste is prepared by mixing a powder comprising Spongilla and water or saline or hydrogen peroxide.

13. The composition of any one of claims 1-12, wherein the first composition is packaged prior to use.

14. The composition of claim 13, wherein the first composition is prepared by heating to at least about 70° C. prior to being packaged.

15. The composition of claim 14, wherein the first composition is heated to at least about 70° C. for at least about 5 minutes prior being packaged.

16. The composition of claim 13, wherein the first composition is prepared by treating with gamma radiation or heat prior to being packaged.

17. The composition of claim 13, wherein the first composition is prepared by treating with gamma radiation or heat after being packaged.

18. The composition of any one of claims 1-17, wherein the Spongilla is Spongilla lacustris.

19. The composition of any one of claims 1-18, wherein the dermal filler comprises one or more of a polymer or co-polymer of hyaluranonic acid, calcium hydroxylapatite, polyalkylimide, polymethylmethacrylate, collagens, polymethyl-methacrylate microspheres (PMMA), polylactic acid, polycaprolactone, carboxymethyl cellulose, poly-L-lactic acid, and cross-linking reagent.

20. The composition of any one of claims 1-19, wherein the dermal filler is a topical derm filler comprising glycerin, peptides derived from collagen, alguronic acid, low molecular weight hyaluronic acid polymers, sodium aceylated hyaluronate, glycolic acid, lactic acid, L-lactic acid, amino acids, NIA-114™, an acetyl peptide, Activen™ XEP-18, and/or Erasa™ XEP-30.

21. The composition of any one of claims 1-19, wherein said derm filler is selected from Restylane®, Restylane® Lyft (Perlane®-L), Restylane® Refyne, Restylane® Defyne, and/or Restylane® Silk, Captique™, Belotero® Hydro, Belotero® Soft, Belotero® Balance, and/or Belotero® Balance, Esthelis™, Fortelis™, Modelis™, Elevess® (Hydrelle®), Hylaform® (hylan B gel), Puragen™, Prevelle® Silk, Radiesse® (+), Radiesse®, Aquamid®, Sculptra® Aesthetic, Bellafill®, and Juvaderm®.

22. The composition of any one of claims 1-21, wherein the second composition comprises deoxycholic acid.

23. The composition of any one of claims 1-22, wherein the second composition is in the form of a solution, an aqueous solution, a powder, or a hydrogel.

24. A method for administering a dermal filler into the skin of a subject, comprising applying to the skin of the subject a first composition comprising Spongilla, and a second composition comprising an effective amount of a dermal filler.

25. The method of claim 24, wherein the Spongilla is in the form of a powder.

26. The method of claim 25, wherein the powder comprises particles that are substantially uniform in size.

27. The method of any one of claims 24-26, wherein not less than 50% of the particles comprising the Spongilla powder pass through a US 70-mesh screen.

28. The method of any one of claims 24-26, wherein the particles comprising the Spongilla powder have an average length of from about 50 μm to about 500 μm.

29. The method of any one of claims 24-26, wherein the particles comprising the Spongilla powder have an average diameter of from about 5 μm to about 50 μm.

30. The method of any one of claims 24-29, wherein the particles comprising the Spongilla powder have an aspect ratio of from about 1 to 100.

31. The method of any one of claims 24-30, wherein the first composition has a residual moisture content of not more than about 10%.

32. The method of any one of claims 24-32, wherein the amount of the first composition comprising Spongilla is from about 0.5 grams to about 50 grams.

33. The method of any one of claims 24-32, wherein the first composition is applied to the skin of the subject in the form of a paste or gel.

34. The method of claim 33, wherein the paste further comprises water or saline or hydrogen peroxide.

35. The method of any one of claim 24 or 34, wherein the paste is prepared by mixing a powder comprising Spongilla and water or saline or hydrogen peroxide.

36. The method of any one of claims 33-35, wherein applying the first composition comprises massaging the paste into a region of the skin of a subject.

37. The method of any one of claims 24-36, wherein said dermal filler comprises one or more of a polymer or co-polymer of hyaluranonic acid, calcium hydroxylapatite, polyalkylimide, polymethylmethacrylate, collagens, polymethyl-methacrylate microspheres (PMMA), polylactic acid, polycaprolactone, carboxymethyl cellulose, poly-L-lactic acid, and cross-linking reagent.

38. The method of any one of claims 24-35, wherein said derm filler is selected from Restylane®, Restylane® Lyft (Perlane®-L), Restylane® Refyne, Restylane® Defyne, and/or Restylane® Silk, Captique™, Belotero® Hydro, Belotero® Soft, Belotero® Balance, and/or Belotero® Balance, Esthelis™, Fortelis™, Modelis™, Elevess® (Hydrelle®), Hylaform® (hylan B gel), Puragen™, Prevelle® Silk, Radiesse® (+), Radiesse®, Aquamid®, Sculptra® Aesthetic, Bellafill®, and Juvaderm®.

39. A method for treating or preventing a skin condition or disease in a subject comprising administering a therapeutically or prophylactically effective amount a first composition comprising Spongilla, and a second composition comprising a therapeutically effective amount a dermal filler to an intradermal compartment of the subject's skin by applying said first and second compositions to the skin of the subject.

40. The method of claim 39, wherein the skin condition or disease is an abnormality of the skin.

41. The method of claim 40, wherein the abnormality of the skin is due to an underlying medical condition.

42. The method of claim 41, wherein the underlying medical condition is lipoatrophy.

43. The method of claim 41, wherein the lipoatrophy is due to HIV or scarring.

44. The method of claim 41, wherein the skin abnormality is rhytids.

45. The method of claim 41, wherein the skin abnormality is skin folds.

46. The method of claim 45, wherein folds includes fine wrinkles in the face, lips, nose, under-eye region, eye brow, head, chin, neck, ear, ear lobes, décolletage, elbow, hand, foot, and/or knee regions.

47. The method of claim 45, wherein folds includes moderate wrinkles of skin in the face, lips, nose, under-eye region, eye brow, head, chin, neck, ear, ear lobes, décolletage, elbow, hand, foot, and knee regions.

48. The method of claim 45, wherein folds includes severe wrinkles or folds of skin in the face, lips, nose, under-eye region, eye brow, head, chin, neck, ear, ear lobes, décolletage, elbow, hand, foot, and knee regions.

49. The method of claim 39, wherein the skin abnormality is skin depressions.

50. The method of any one of claims 39-49, wherein the skin condition is treated by augmentation of an exisiting body part.

51. The method any one of claims 39-50, wherein the skin condition is treated by improving nasal function.

52. The method of any one of claims 39-51, wherein the intradermal compartment of the subject's skin includes regions of the face, lips, nose, under-eye region, eye brow, head, chin, neck, ear, ear lobes, décolletage, elbow, hand, foot, and knee regions and wherein administration of the first and second compositions results in modification, augmentation, and/or correction of the regions to which the composition is applied.

53. The method of any one of claims 39-52, wherein the Spongilla is in the form of a powder.

54. The method of claim 53, wherein the powder comprises particles that are substantially uniform in size.

55. The method of any one of claims 52-54, wherein not less than 50% of the particles comprising the Spongilla powder pass through a US 70-mesh screen.

56. The method of any one of claims 52-54, wherein the particles comprising the Spongilla powder have an average length of from about 50 μm to about 500 μm.

57. The method of any one of claims 52-54, wherein the particles comprising the Spongilla powder have an average diameter of from about 5 μm to about 50 μm.

58. The method of any one of claims 52-54, wherein the particles comprising the Spongilla powder have an aspect ratio of from about 1 to 100.

59. The method of any one of claims 39-58, wherein the first composition has a residual moisture content of not more than about 10%.

60. The method of any one of claims 39-59, wherein the amount of the first composition comprising Spongilla is from about 0.5 grams to about 50 grams.

61. The method of any one of claims 39-60, wherein the first composition is applied to the skin of the subject in the form of a paste.

62. The method of claim 61, wherein the paste further comprises water or saline or hydrogen peroxide.

63. The method of any one of claim 61 or 62, wherein the paste is prepared by mixing a powder comprising Spongilla and water or saline or hydrogen peroxide.

64. The method of any one of claims 39-63, wherein applying the first composition comprises massaging the paste into a region of the skin of a subject.

65. The method of any one of claims 39-64, wherein said dermal filler comprises one or more of a polymer or co-polymer of hyaluranonic acid, calcium hydroxylapatite, polyalkylimide, polymethylmethacrylate, collagens, polymethyl-methacrylate microspheres (PMMA), polylactic acid, polycaprolactone, carboxymethyl cellulose, poly-L-lactic acid, and cross-linking reagent.

66. The method of any one of claims 39-65, wherein said dermal filler is a topical derm filler comprising glycerin, peptides derived from collagen, alguronic acid, low molecular weight hyaluronic acid polymers, sodium aceylated hyaluronate, glycolic acid, lactic acid, L-lactic acid, amino acids, NIA-114™, an acetyl peptide, Activen™ XEP-18, and/or Erasa™ XEP-30.

67. The method of any one of claims 39-65, wherein said derm filler is selected from Restylane®, Restylane® Lyft (Perlane®-L), Restylane® Refyne, Restylane® Defyne, and/or Reslylane® Silk, Captique™, Belotero® Hydro, Belotero® Soft, Belotero® Balance, and/or Belotero® Balance, Esthelis™, Fortelis™, Modelis™, Elevess® (Hydrelle®), Hylaform® (hylan B gel), Puragen™, Prevelle® Silk, Radiesse® (+), Radiesse®, Aquamid®, Sculptra® Aesthetic, Bellafill®, and Juvaderm®.

68. The method of any one of claims 39-67, wherein said second composition comprises deoxycholic acid.

69. A method for enhancing skin permeation of a topically applied dermal filler composition in a skin of a subject, comprising applying to the skin a first composition comprising Spongilla, followed by applying a second composition comprising an effective amount of a dermal filler.

70. The method of claim 69, wherein the Spongilla is in the form of a powder.

71. The method of claim 70, wherein the powder comprises particles that are substantially uniform in size.

72. The method of any one of claims 69-71, wherein not less than 50% of the particles comprising the Spongilla powder pass through a US 70-mesh screen.

73. The method of any one of claims 69-72, wherein the particles comprising the Spongilla powder have an average length of from about 50 μm to about 500 μm.

74. The method of any one of claims 69-73, wherein the particles comprising the Spongilla powder have an average diameter of from about 5 μm to about 50 μm.

75. The method of any one of claims 69-74, wherein the particles comprising the Spongilla powder have an aspect ratio of from about 1 to 100.

76. The method of any one of claims 69-75, wherein the first composition has a residual moisture content of not more than about 10%.

77. The method of any one of claims 69-76, wherein the amount of the first composition comprising Spongilla is from about 0.5 grams to about 50 grams.

78. The method of any one of claims 69-77, wherein the first composition is applied to the skin of the subject in the form of a paste.

79. The method of claim 78, wherein the paste further comprises water or saline or hydrogen peroxide.

80. The method of any one of claim 78 or 79, wherein the paste is prepared by mixing a powder comprising Spongilla and water or saline or hydrogen peroxide.

81. The method of any one of claims 66-77, wherein applying the first composition comprises massaging the paste into a region of the skin of a subject.

82. The method of any one of claims 69-81, wherein said dermal filler comprises one or more of a polymer or co-polymer of hyaluranonic acid, calcium hydroxylapatite, polyalkylimide, polymethylmethacrylate, collagens, polymethyl-methacrylate microspheres (PMMA), polylactic acid, polycaprolactone, carboxymethyl cellulose, poly-L-lactic acid, and cross-linking reagent.

83. The method of any one of claims 69-82, wherein said dermal filler is a topical dermal filler comprising glycerin, peptides derived from collagen, alguronic acid, low molecular weight hyaluronic acid polymers, sodium aceylated hyaluronate, glycolic acid, lactic acid, L-lactic acid, amino acids, NIA-114™, an acetyl peptide, Activen™ XEP-18, and/or Erasa™ XEP-30.

84. The method of any one of claims 69-83, wherein the second composition comprises deoxycholic acid.

85. The method of any one of claims 24-84, wherein the first composition is applied to the skin of the subject prior to the second composition being applied to the skin of the subject.

86. The method of claim 85, wherein the first composition is applied to the skin of the subject and is permitted to dry on the skin of the subject prior to application of the second composition to the skin of the subject.

87. The method of any one of claim 81 or 86, wherein the first composition is washed off the skin of the subject prior to the second composition being applied to the skin of the subject.

88. The method of any one of claim 24, 39 or 69, wherein the first composition and the second composition are mixed together and the resulting mixture is applied to the skin of the subject.

89. The method of any one of claims 24-88, wherein the first composition and the second composition are applied to the skin of the subject no more than once every 3 months.

90. The method according to any one of claims 24-89, wherein the first composition comprising Spongilla is applied to the skin of the subject at least once per week for at least one week.

91. The method according to any one of claims 23-91, wherein the skin of the subject is cleaned using a non-comedogenic cleanser, water, or a combination of a non-comedogenic cleanser and water following application of the first composition comprising Spongilla.

92. The method according to any one of claims 24-91, wherein the skin of the subject is cleaned using a non-comedogenic cleanser, water, or a combination of a non-comedogenic cleanser and water following application of the second composition to the skin of the subject.

93. The method of any one of claims 24-92, wherein the second composition is in the form of a hydrogel.

94. A kit comprising (a) a first composition comprising Spongilla, and (b) a second composition comprising one or more dermal fillers in an amount effective to treat a skin condition in a subject

95. A kit comprising the kit of claim 94 and a reconstitution reagent.

96. A kit comprising the kit of claim 94 and an antiseptic reagent.

97. A kit comprising the kit of claim 94 and a reconstition reagent, and an antiseptic reagent.

98. The kits of claims 94-97, further comprising instructions.
